(19) United States
(12) Patent Application Publication
(10) Pub. No.: US 2014/0017701 A1
(43) Pub. Date: Jan. 16, 2014

(54) CHIMERIC PDK1 KINASES

(71) Applicants: Ricardo M. Biondi, Frankfurt/Main (DE); Laura A. L. Lopez Garcia, Zurich (CH); Jörg O. Schulze, Frankfurt/Main (DE)

(72) Inventors: Ricardo M. Biondi, Frankfurt/Main (DE); Laura A. L. Lopez Garcia, Zurich (CH); Jörg O. Schulze, Frankfurt/Main (DE)

(21) Appl. No.: 14/001,539

(22) PCT Filed: Feb. 24, 2012
(86) PCT No.: PCT/EP2012/053183

§ 371 Date: Sep. 30, 2013

(30) Foreign Application Priority Data
Feb. 24, 2011 (EP) 11155859.9
Aug. 19, 2011 (EP) 11178105.0

(51) Int. Cl. C12N 9/12; G01N 33/573

(52) U.S. Cl. CPC C12N 9/12; G01N 33/573
USPC 435/7.4; 435/194; 536/23.2; 435/320.1; 548/310.1; 435/369; 435/348

(57) ABSTRACT

The invention provides chimeric 3-phosphoinositide-dependent protein kinase 1 (PDK1), the PIF-binding pocket of which has mutations to mimic a second protein kinase, its production and use. The invention further provides a method for screening for compounds interacting with the PIF-pocket of an AGC kinase.

The invention provides chimeric 3-phosphoinositide-dependent protein kinase 1 (PDK1), the PIF-binding pocket of which has mutations to mimic a second protein kinase, its production and use. The invention further provides a method for screening for compounds interacting with the PIF-pocket of an AGC kinase.

### BACKGROUND OF THE INVENTION

In general, drug design projects benefit immensely if crystal structures of the target protein with bound compounds are available. Analysis of the binding mode will give valuable feedback on how to improve the compounds to have more interactions with the protein; it may also lead to compounds with higher specificity to their target protein. Nevertheless, the crystallization process is the bottleneck in crystallography; it is unpredictable and needs a large amount of pure and stable protein for random screening of hundreds of conditions. In our case, we are trying to crystallize the catalytic domain of PKCζ for 1.5 years already. Unfortunately, this protein is aggregating/oligomerizing almost completely and there are also issues with heterogeneous phosphorylation. Our crystallization efforts are continuing with new constructs.

As part of our ongoing research, we identified low-molecular-weight compounds that inhibit PKCζ and target a hydrophobic pocket of protein kinase C zeta type (PKCζ) that resembles the so-called PIF-binding pocket of PDK1. At this stage, we could not improve the compounds significantly without knowing the exact binding mode. Thus, feedback from crystal structures was necessary to speed up the process of developing compounds with higher affinity.

In WO2010/043719 we characterized certain mutants of PDK1, notably a double mutant (dm) of the PDK1 catalytic domain (PDK1 50-359 [Y288G Q292A], SEQ ID NO:3) that crystallized and allowed small molecules to bind to the PIF-binding pocket in the crystal with outstanding resolution of up to 1.25 A. Although the catalytic domains of PDK1 and PKCζ share only 25% sequence identity, they both belong to the sub-family of AGC kinases and share a common and very conserved fold (as indicated by the crystal structure of closely related PKCₗ in complex with the inhibitor BIM1 (PDB-entry 1zrz)).

### SUMMARY OF THE INVENTION

Thus, it was now found that mutating the PIF-binding pocket of PDK1 to mimic that of other kinases such as PKCζ, i.e. modifying only the binding site to allow binding of PKCζ-specific inhibitors, allowed the analysis of the binding mode by crystallography. The chimera proteins still possess the properties of PDK1dm: excellent production yield in insect cells, the established purification protocol can be applied and—most importantly—it readily crystallized at the same crystallization conditions as PDK1dm (plus the PIF-binding pocket is accessable for soaking of compounds). The invention thus provides:

(1) A chimeric 3-phosphoinositide-dependent protein kinase 1 (PDK1) having the PDK1 hydrophobic pocket in the position equivalent to the hydrophobic PIF-binding pocket defined by the residues Lys115, Ile118, Ile119, Val124, Val127 and/or Leu155 of full length human PDK1 shown in SEQ ID NO:2 and having the phosphate binding pocket equivalent to the phosphate binding pocket defined by the residues Lys76, Arg131, Thr148 and/or Gln150 of full length hPDK1 shown in SEQ ID NO:2, wherein said mutant protein kinase has a at least two mutations in one of its motives equivalent to AGNEYLIFQK (SEQ ID NO:54) and LDHPFFVK (SEQ ID NO:55) of hPDK1, or a fragment or derivate thereof and wherein the PIF-binding pocket has mutations to mimic a second protein kinase.

(2) A preferred embodiment of aspect (1) above, wherein the chimeric PDK1 is derived from a truncated double mutant (dm) of the hPDK1 (PDK1₅₀₋₃₅₉ [Y288G Q292A], SEQ ID NO:3) having the mutations Tyr288 Gly and Gln292Ala.

(3) A polynucleotide sequence encoding the chimeric PDK1 of (1) or (2) above.

(4) A vector comprising the polynucleotide sequence of (3) above.

(5) A host cell transformed with the vector of (4) above and/or comprising the polynucleotide sequence of (3) above.

(6) A process for producing the chimeric PDK1 of (1) or (2) above which comprises culturing the host cell of (5) above and isolating said chimeric PDK1.

(7) A method for identifying a compound that binds to the PIF-binding pocket allosteric site mimicked by the chimeric PDK1 protein kinase as defined in (1) or (2) above, which comprises the step of determining the effect of the compound on the chimeric PDK1 of (1) or (2) above or the ability of the compound to bind to said chimeric PDK1.

(8) A kit for performing the method of (7) above which comprises a chimeric PDK1 of (1) or (2) above.

(9) A compound identified by the method of (7) above binding to the PIF-binding pocket allosteric site of the chimeric PDK1.

(10) A method for screening for a compound that interacts with the PIF-pocket of an AGC kinase, which method comprises the step of determining the effect of the compound to be tested on the interaction between a first protein comprising the PIF-pocket of said AGC kinase and a second protein comprising the C1-domain of same or different AGC kinase.

(11) A kit for performing the method of (10) above which comprises first and second proteins as defined in (10) above.

(12) A compound identified by the method of (10) above binding to the PIF-binding pocket of an AGC kinase.

### SHORT DESCRIPTION OF THE FIGURES

FIG. 1: Partial sequence alignments of human 3-phosphoinositide-dependent protein kinase 1 (PDK1) with (A) human protein kinase C zeta type (PKC); (B) human protein kinase C iota type (PKCₗ); (C) Candida albicans PKH1(PKH1); (D) human serine/threonine-protein kinase N2, a.k.a. protein-kinase C-related kinase 2 (PRK2); (E) human serine/threonine-protein kinase Sgk1, a.k.a. serum/glucocorticoid-regulated kinase 1 (SGK1); (F) human ribosomal protein S6 kinase beta-1, a.k.a. 70 kDa ribosomal protein S6 kinase 1 (S6K1); (G) human RAC-alpha serine/threonine-protein kinase, a.k.a protein kinase B alpha (PKBα), a.k.a. protein kinase Akt-1 (AKT1); (H) Human RAC-beta serine/threonine-protein kinase, a.k.a protein kinase B beta (PKBβ), a.k.a. protein kinase Akt-2 (AKT2); (I) Human ribosomal protein S6 kinase alpha-3, a.k.a. ribosomal S6 kinase 2 (RSK2); and (J) Human ribosomal protein S6 kinase alpha-5, a.k.a. mitogen- and stress-activated protein kinase 1 (MSK1). The bold residues are the mutation sites for the PDK1.

FIG. 2: Compounds PS168 and PS172; activity assays with the PDK1/PKCζchimera, that had similar basal activity as the non-mutated PDK1 50-359 counterpart, indicating that the protein was well folded and that the mutations did not affect its activity towards PDK1 peptide substrate. Most importantly, while PDK1 is activated by compounds PS168 and PS172, PDK1/PKCζchimera is inhibited, similarly to PKCζ wild type (wt).

FIG. 3: (A) PS168; (B) PS315 and (C) PIF-binding pocket with mutated residues.

FIG. 4: GST-PKCζ, as indicated in the figure. After 48 h the cells were lysed, the GST-fusion protein purified by affinity chromatography on glutathione sepharose beads, the product electrophoresed on a 10% SDS-polyacrylamide gel and immune-blotted using an anti-GST antibody to detect pulled down GST-PKCζ and an anti-Myc antibody to detect co-purified Myc-PDK1. The crude extract was used to estimate the total amount of Myc-PDK1 expressed in the cells. The experiment did not reveal any effect of the N-terminal domains of PKCζ on the interaction with PDK1. Duplicates of the transfection of each condition are shown.

FIG. 5: Phosphorylation state of PKCζ deletion constructs and mutants. (a) Immunoblot of purified GST-PKCζ deletion constructs and mutants using anti-phospho activation loop antibody. (b) Immunoblot of purified GST-PKCζ deletion constructs and mutants using anti-phospho Z/turn-motif antibody. The extent of phosphorylation was quantified using the program MultiGauge V3.0 (Fujifilm) and normalized over the amount of loaded protein. A value of 1 was assigned to the phosphorylation of wild type GST-PKCζ 1-592.

FIG. 6: Thermal stability of PKCζ and PKCζ [7R/K-A]. The wild type (wt) GST-PKCζ (∘) or the GST-PKCζ [7Arg/Lys-Ala] (7R/K-A) mutant (□) were incubated in the presence (closed symbols , ▪) or absence (open symbols ∘, □) of lipid activator (LA), incubated for 2 min at the indicated temperatures and assayed for remaining protein kinase activity at 24° C. using MBP as substrate. The activity of PKCζ or PKCζ [7R/K-A] obtained by incubation at 24° C. was set as 100%. (a) PKCζ [7R/K-A] was significantly less stable in 2 min temperature shift than PKCζ wt. (b) The presence of LA destabilized PKCζ wt. (c) PKCζ [7R/K-A] did not lose further protein stability in the presence of lipids, indicating that the pseudosubstrate region mediated the LA-dependent loss of thermal stability. The assay shown was performed in triplicates with similar results obtained in two separate experiments.

FIG. 7: Effect of PSRtide on the activity of PKCζ. (a) The activity of GST-PKCζ wt and deletion constructs was measured using 100 μM of PSRtide as the substrate of the reaction in the presence (gray columns) or absence (white columns) of 100 ng of phosphatidylserine (LA). (b-f) Models that explain the results observed in (a). (b) PKCζ 1-592 in the presence of PSRtide had high basal activity and was not further activated by LA. PSRtide competed and displaced the PSR of PKCζ, displacing as well the N-terminal domains. (c) PKCζ Δ98 had the same behavior as 1-592 towards PSRtide. (d) PKCζ Δ129, which lacks the PSR, had much lower basal activity than the other constructs indicating that PKCζ Δ129 is in an inactive conformation. This is consistent with the idea that the binding of PSRtide to the substrate binding site of PKCζ Δ129 cannot remove the C1 domain interaction with the catalytic domain. This result can be explained that apparently the displacement of the PSR is necessary to displace the C1 allosteric inhibition. (e and f) PKCζ Δ180 and PKCζ Δ240 had similar basal activity to PKCζ 1-592.

FIG. 8: Effect of PS168 and PS171 on PKCζ using PSRtide as the substrate of the reaction. The effect of PS168 and PS171 on the activity of (a) the full length (1-592) and (b) the catalytic domain (Δ240) of PKCζ is shown.

FIG. 9: Effect of compounds on PKCζ-dependent NFκB activation. Pre-incubation with PS168 and PS171 inhibits PKCζ-dependent NFκB activation in U937 cells (IC50<50 μM). In contrast, PS153, an analogue compound that is inactive in vitro, had no effect on NFκB activation by TNFα. Incubation of the cells at each concentration of compounds was performed in triplicates. A representative experiment of three is shown.

FIG. 10: Molecular mechanism of regulation of PKCζ by N-terminal domains. (A) Structure of the catalytic core of PKCζ (model based on PKCₗ structure, PDB code 1ZRZ, Messerschmidt et al., 2005, J. Mol. Biol. 352, 4, 918-931). (B) Schematic overview of PKC isoforms indicating the different domains present in the classical, novel and atypical PKCs (C2, C2 domain; PSR, pseudosubstrate region; C1, C1 domain; PB1, PB1 domain; Cat. Domain, protein kinase catalytic domain) and the PKCζ wild type (1-592) and truncated versions used in this study. (C) Activity of the N-terminally truncated PKCζ constructs in the presence or absence of lipid activators (LA) using MBP as a substrate. A significant increase in the activity of PKCζ was observed after removal of the C1 domain. The average of two independent experiments using two different batches of purified deletion constructs is shown. The specific activity of PKCζ [Δ240] (100%) varied between 25 and 40 nmol/mg min in different purifications.

FIG. 11: Interaction of C1 domain constructs of PKCₗ with its catalytic domain. The AlphaScreen interaction assay shows the binding of GST-PSR-C1 (left y-axis) or GST-C1 (right y-axis) to His-PKCₗ Δ223. The interaction of both, GST-PSR-C1 and GST-C1, with His-PKCₗ Δ223 was strongly diminished upon addition of the HM-peptide derived from the AGC-kinase ROCK (ROCK-HM). In contrast, the corresponding peptide phosphorylated at the HM phosphorylation site (ROCK-pHM) was not able to displace the binding, indicating a high degree of selectivity.

FIG. 12: Binding of the benzimidazole compounds to the PIF-pocket of different AGC kinases can allosterically activate or inhibit the kinases, acting as agonists or antagonists of the activity. (A) Activation of PDK1 by PS114. Crystal structures of PDK1 in complex with the allosteric activators PS114 (B) and PS171 (C). The ring systems of both compounds are positioned similarly in the PIF-binding pocket of PDK1. The carboxylate moiety of PS114 is unresolved and is shown in transparent white. The depicted 2Fₒ-F_{c} electron density maps are contoured at 1 s. (D) Activity assays with PKCζ mutants identify the PIF-pocket as the target site of PS168 and PS171. PS168 and PS171 (50 μM) inhibit PKCζ wt but not PKCₗ or PKCζ proteins mutated within the PIF-pocket (PKCζ [Leu328Phe] and PKCζ [Val297Leu]). PS168 activates PDK1 wt but inhibits PDK1[P-P-ζ], indicating that the replacement of the PIF-binding pocket amino acids with those of PKCζ changes the conformational transition from activation to inhibition by compounds targeting the same site.

FIG. 13: Structural models showing the active and inactive states of PKCζ in accordance with the observed biochemical data. Models of the individual domains were generated using SWISS-MODEL. (A) Model of the active state. The hydrophobic motif (red) binds in the PIF-pocket of the catalytic domain of PKCζ (yellow; based on PDB code 1ZRZ). (B) Model of the inactive state. C1 domain (orange) and PSR (blue, both based on PDB code 2ENN) bind to the catalytic domain and inhibit its activity. PSR was placed so that Ala119, the residue mutated to a phosphorylatable Ser in the peptide PSRtide, is in the location commonly observed for substrates of AGC protein kinases. The N- to C-terminal direction in the image is from the left to right as e.g. observed for the crystal structure of PKA in complex with peptide inhibitor PKI (PDB code 1ATP). By using these constraints, the C1 domain is consequentially placed close to alpha-helix C of the PIF-pocket.

FIG. 14: Photo of crystals of the PDK1/PKCₗ chimera grown in the crystallization conditions of PDK1_{dm} crystal form II.

FIG. 15: PIF-pocket of a crystal structure of the PDK1/PKCₗ chimera soaked with compound PS267. Residues mutated to mimic the PKCₗ PIF-pocket are highlighted in orange. The |2Fₒ−F_{c}| electron density of PS267 is contoured at 1σ.

FIG. 16: Photo of crystals of the PDK1/SGK chimera grown in the crystallization conditions of PDK1_{dm} crystal form II.

FIG. 17: PIF-pocket of a crystal structure of the PDK1/SGK chimera soaked with compound PS238. Residues mutated to mimic the SGK PIF-pocket are highlighted in orange. The |2Fₒ−F_{c}| electron density of PS238 is contoured at 1σ.

FIG. 18: Photo of crystals of the PDK1/PRK2 chimera grown in the crystallization conditions of PDK1_{dm} crystal form II.

FIG. 19: PIF-pocket of a crystal structure of the PDK1/PRK2 chimera. Residues mutated to mimic the PRK2 PIF-pocket are highlighted in orange.

FIG. 20: Photo of crystals of the PDK1/PKBα chimera grown in the crystallization conditions of PDK1_{dm} crystal form II upon addition of the additive CYMAL.

FIG. 21: PIF-pocket of a crystal structure of the PDK1/PKBα chimera. Residues mutated to mimic the PKBα PIF-pocket are highlighted in orange.

Sequence Listing - Free Text

| SEQ | |
|---|---|
| ID NO: | Description |
| 1/2 | full length human 3-phosphoinositide-dependent protein kinase |
| | 1 (PDK1) |
| 3 | Y288G, Q292A hPDK1₅₀₋₃₅₉ (PDK1_{dm}) |
| 4/5 | human protein kinase C zeta type (PKCζ) |
| 6/7 | PDK1 and PKCζ fragments |
| 8 | PDK1_{dm}(50-359)-PKCζ-chimera with His-tag |
| 9/10 | human protein kinase C iota type (PKCι) |
| 11/12 | PDK1 and PKCι fragments |
| 13 | PDK1_{dm}(50-359)-PKCι-chimera with His-tag |
| 14/15 | Candida albicans PKH1 |
| 16/17 | PDK1 and PKH1 fragments |
| 18 | PDK1_{dm}(50-359)-PKH1-chimera with His-tag |
| 19/20 | human serine/threonine-protein kinase N2, a.k.a. protein- |
| | kinase C-related kinase 2 (PRK2) |
| 21/22 | PDK1 and PRK2 fragments |
| 23 | PDK1_{dm}(50-359)-PRK2-chimera with His-tag |
| 24/25 | human serine/threonine-protein kinase Sgk1, a.k.a. |
| | serum/glucocorticoid-regulated kinase 1 (SGK1) |
| 26/27 | PDK1 and SGK1 fragments |
| 28 | PDK1_{dm}(50-359)-SGK1-chimera with His-tag |
| 29/30 | human ribosomal protein S6 kinase beta-1, a.k.a. 70 kDa |
| | ribosomal protein S6 kinase 1 (S6K1) |
| 31/32 | PDK1 and S6K1 fragments |
| 33 | PDK1_{dm}(50-359)-S6K1-chimera with His-tag |
| 34/35 | human RAC-alpha serine/threonine-protein kinase, a.k.a protein |
| | kinase B alpha (PKBα), a.k.a. protein kinase Akt-1 (AKT1) |
| 36/37 | PDK1 and AKT1 fragments |
| 38 | PDK1_{dm}(50-359)-AKT1-chimera with His-tag |
| 39/40 | human RAC-beta serine/threonine-protein kinase, a.k.a protein |
| | kinase B beta (PKBβ), a.k.a. protein kinase Akt-2 (AKT2) |
| 41/42 | PDK1 and AKT2 fragments |
| 43 | PDK1_{dm}(50-359)-AKT2-chimera with His-tag |
| 44/45 | human ribosomal protein S6 kinase alpha-3, a.k.a. ribosomal |
| | S6 kinase 2 (RSK2) |
| 46/47 | PDK1 and RSK2 fragments |
| 48 | PDK1_{dm}(50-359)-RSK2-chimera with His-tag |
| 49/50 | human ribosomal protein S6 kinase alpha-5, a.k.a. mitogen- |
| | and stress-activated protein kinase 1 (MSK1) |
| 51/52 | PDK1 and MSK1 fragments |
| 53 | PDK1_{dm}(50-359)-MSK1-chimera with His-tag |
| 54/55 | Motives of human PDK1 |
| 56 | substrate for PKCζ |
| 57 | substrate for PDK1 |
| 58 | fragment of PDK1-PKCζ chimera |
| 59/60 | substrates for AlphaScreen interaction assay |

### DETAILED DESCRIPTION OF THE INVENTION

In a preferred embodiment of aspect (1) of the invention the chimeric PDK1 (hereinafter shortly referred to as “chimeric PDK1 of the invention” or “PDK1 chimera of the invention”) is a mammalian protein kinase, preferably is derived from the hPDK1 having SEQ ID NO:2. Furthermore it is preferred that the mutation in the motif of SEQ ID NO:54 is a non-conservative mutation, and/or is a mutation of the residues Y or Q. Particularly preferred is that said motif has the mutation of the residue Y with G or a mutation of the residue Q to A, most preferred is that said motif has the Y to G and Q to A mutations. Also it is preferred that the mutation in the motif of SEQ ID NO:55 is a non-conservative mutation, and/or is a mutation of the residues D, H, P, or K. Particularly preferred is that said motif has the mutation of the residue D or K with M, H or P.

In another preferred embodiment of aspect (1) the chimeric PDK1 is derived from hPDK1 shown in SEQ ID NO:2 and has at least two mutations at a position corresponding to positions Tyr288 and Gln292, and may have one or more further point mutations at positions corresponding to Lys296 and Ile295, wherein the numbering refers to the full length hPDK1 shown in SEQ ID NO:2. Particularly preferred is that the chimeric PDK1 has the mutations Tyr288 Gly and Gln292Ala, wherein the numbering refers to the full length hPDK1 shown in SEQ ID NO:2.

Still in another preferred embodiment of aspect (1) the chimeric PDK1 is derived from a fragment of the chimeric PDK1 protein kinase that is C- and/or N-terminally truncated and comprises the hydrophobic PIF-binding pocket, the phosphate binding pocket and the motives of SEQ ID NOs:54 and 55. Particularly preferred is that the fragment comprises the residues corresponding to 50-359 or 67-359 of hPDK1 shown in SEQ ID NO:2. Most preferred is that the chimeric PDK1 protein kinase is derived from the truncated double mutant (dm) of the hPDK1, namely PDK1₅₀₋₃₅₉ [Y288G Q292A], SEQ ID NO:3 (aspect (2) of the invention).

In a preferred embodiment of aspects (1) and (2) of the invention, the second protein kinase that is mimicked by the PIF pocket of the chimeric PDK1 is a mammalian protein kinase grouped within the AGC group of protein kinases, such as SGK, PKB, S6K, MSK, RSK, LAT, NDR, MAST, ROCK, DMPK, MRCK, PKA, PKG, GRK, PRK, PKC and their isoforms, or Aurora or YANK protein kinases and their isoforms, or is a protein kinases from infectious organisms such as Candida species including Candida albicans, Aspergillus spp., Cryptococcus neoformans, Histoplasma capsulatum, or Coccidioides. Particularly preferred second protein kinases that are mimicked by the PIF pocket of the chimeric PDK1 include a human protein kinase C zeta type (hPKCζ), a human protein kinase C iota type (hPKCₗ), a Candida albicans PKH1, a human serine/threonine-protein kinase N2, a.k.a. protein-kinase C-related kinase 2 (hPRK2), a human serine/threonine-protein kinase Sgk1 (a.k.a. serum/glucocorticoid-regulated kinase 1; hSGK1), a human ribosomal protein S6 kinase beta-1 (a.k.a. 70 kDa ribosomal protein S6 kinase 1; hS6K1), a human RAC-alpha serine/threonine-protein kinase (a.k.a protein kinase B alpha (PKBα), a.k.a. protein kinase Akt-1; hAKT1), a human RAC-beta serine/threonine-protein kinase (a.k.a protein kinase B beta (PKBβ), a.k.a. protein kinase Akt-2; hAKT2), a human ribosomal protein S6 kinase alpha-3 (a.k.a. ribosomal S6 kinase 2; hRSK2) and a human ribosomal protein S6 kinase alpha-5 (a.k.a. mitogen- and stress-activated protein kinase 1; hMSK1).

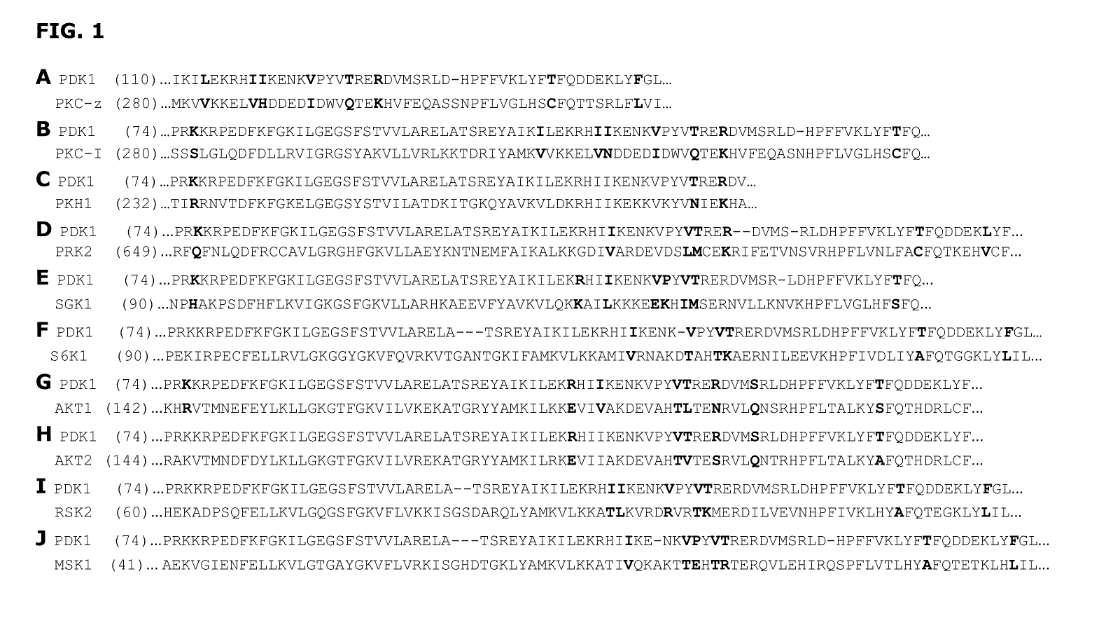

The PDK1 chimera were constructed according to sequence and structural alignments and care was taken not to modify the more “vital” inner core of PDK1 like the active site or residues likely to relay conformational changes induced by the allosteric compounds (see FIG. 1).

Concerning the chimeric PDK1 that mimics the PIF pocket of hPKCζ (SEQ ID NO:5) the differences between hPDK1 and hPKCζ are shown in FIG. 1A. Eight mutations were introduced to produce the PDK1/PKCζ chimera (first column: PDK1 numbering; second column: PKCζ numbering):

### Leu113->Val283

### Ile118->Val288

### Ile119->His289

### Val124->Ile294

### Thr128->Gln298

### Arg 131->Lys301

### Thr148->Cys319

### Phe157->Leu294

Thus the PDK1/PKCζ chimera has the mutations Leu113Val, Ile118Val, Ile119H is, Val124Ile, Thr128Gln, Arg131Lys, Thr148Cys and Phe157Leu in its PIF binding pocket (wherein the numbering refers to the full length hPDK1 sequence of SEQ ID NO:2). Particularly preferred is a PDK1/PKCζ chimera that has a sequence comprising amino acid residues 24 to 334 of SEQ ID NO:8.

In the attached experiments evidence is provided that PS168 and PS171 are allosteric inhibitors of PKCζ. However, obtaining the crystal structure of allosteric inhibitors binding to the PIF-pocket would be strong evidence of their binding site. Moreover, this information would facilitate further drug discovery efforts. The results indeed provide structural information that the allosteric inhibitory compounds indeed bind specifically to the PIF-pocket of PKCζ. In an additive screening ammonium sulfate was identified as an additive enhancing crystal size and quality even further. The maximum resolution obtained so far for PDK1/PKCζ chimera was 1.35 Å.

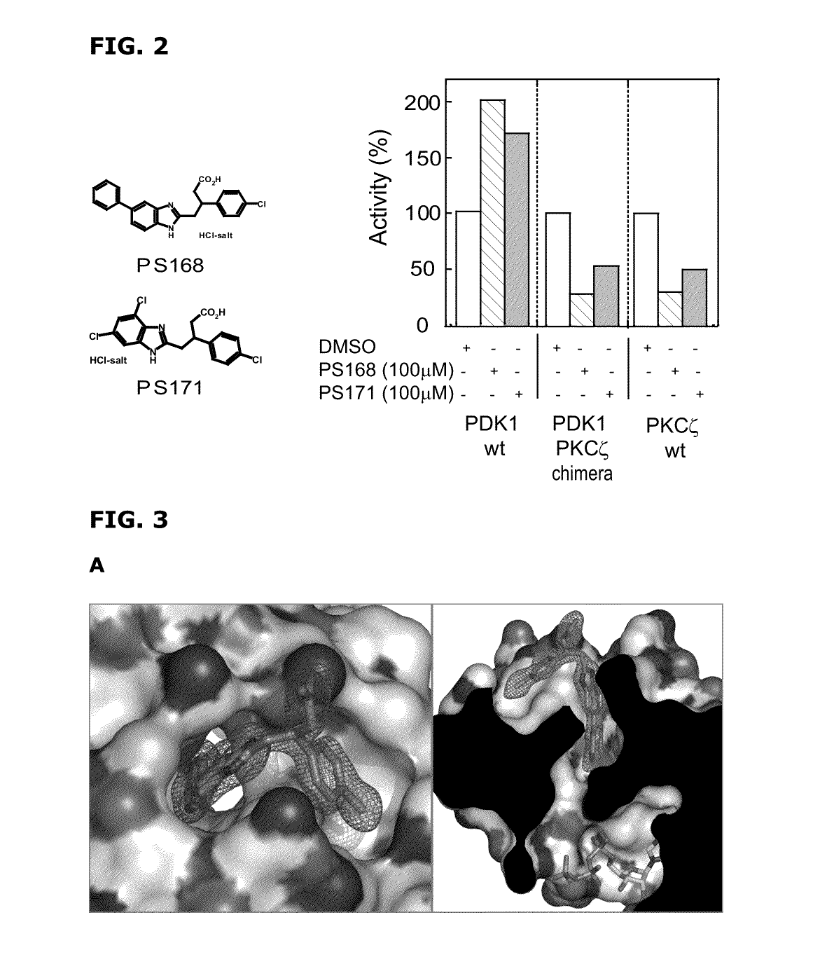

Interestingly, in activity assays, the PDK1/PKCζ chimera had similar basal activity as the non-mutated PDK1 50-359 counterpart, indicating that the protein was well folded and that the mutations did not affect its activity towards PDK1 peptide substrate. Most importantly, while PDK1 is activated by our compounds PS168 and PS172, the chimera is instead inhibited by these compounds, indicating that the mutations at the PIF-binding pocket had indeed affected the binding of the compounds (FIG. 2):

Of note, the when the equivalent mutations were performed on the full length PDK1, the resulting PDK1 1-559/PKCζ chimera did not have such a strong phenotype. Therefore, it is a good surprise that the PDK150-359/PKCζ chimeric construct mimics quite precisely the effect of PS168 and PS171 on PKCζ.

High resolution crystal structures were solved of the PDK150-359/PKCζ chimera apo form and in complex with compounds PS168 (1.35 Å resolution) and PS315 bound to the chimera protein (1.65 Å resolution each). These structures prove unambiguously that our compounds are binding to the mutated binding pocket. Furthermore, all eight mutations intended were verified by these structures.

These crystal structures revealed a new feature to the PIF-binding pocket: the mutations created a much deeper PIF-binding pocket and actually opened up a tunnel to the active site. Homology models based on the PKCₗ structure confirmed that this feature should be very similar in original PKCζ. Intriguingly, compounds PS168 and PS315 share a third phenyl ring as a common feature. This ring was found to be buried deep inside the tunnel and to put strain on the catalytically active residue Lys111 usually hold in place by a strong salt bridge with Glu130. Thus, the structural information generated by the PDK1/PKCζ chimera gave invaluable information about the binding mode and initiated the synthesis of a whole series of compounds targeting specifically the Lys111-Glu130 salt bridge by modifying the third ring.

Concerning the chimeric PDK1 that mimics the PIF pocket of hPKCi (SEQ ID NO:10) the differences between hPDK1 and hPKCₗ are shown in FIG. 1B. Eight mutations were introduced to produce the PDK1/PKCₗ chimera (first column: PDK1 numbering; second column: PKCₗ numbering):

### Lys76->Ser239

### Leu113->Val275

### Ile118->Val281

### Ile119->Asn282

### Val124->Ile287

### Thr128->Gln291

### Arg131->Lys294

### Thr148->Cys312

Thus the PDK1/PKCₗ chimera has the mutations Lys76Ser, Leu113Val, Ile118Val, Ile119Asn, Val124Ile, Thr128Gln, Arg131Lys and Thr148Cys in its PIF binding pocket (wherein the numbering refers to the full length hPDK1 sequence of SEQ ID NO:2). Particularly preferred is a PDK1/PKCₗ chimera that has a sequence comprising amino acid residues 24 to 334 of SEQ ID NO:13.

Concerning the chimeric PDK1 that mimics the PIF pocket of PKH1 (the PDK1 analogue from the pathogen Candida albicans; SEQ ID NO:15) the differences between hPDK1 and PHK1 are shown in FIG. 1C. Three mutations were introduced to produce the PDK1/PKH1 chimera (first column: PDK1 numbering; second column: PHK1 numbering):

### Lys76->Arg234

### Thr128->Asn286

### Arg 131->Lys289

(note: Lys76 does not belong to the PIF-binding pocket per se; nevertheless, this N-terminal residue needs to be mutated too, because it was observed to interact with several activating compounds of PDK1)

Thus the PDK1/PHK1 chimera has the mutations Lys76Arg, Leu128Asn286 and Arg131Lys in its PIF binding pocket (wherein the numbering refers to the full length hPDK1 sequence of SEQ ID NO:2). Particularly preferred is a PDK1/PHK1 chimera that has a sequence comprising amino acid residues 24 to 334 of SEQ ID NO:18.

Concerning the chimeric PDK1 that mimics the PIF pocket of hPRK2 (SEQ ID NO:20) the differences between hPDK1 and hPRK2 are shown in FIG. 1D. Seven mutations were introduced to produce the PDK1/PRK2 chimera (first column: PDK1 numbering; second column: PRK2 numbering):

### Lys76->Gln651

### Ile119->Val694

### Val127->Leu702

### Thr128->Met703

### Arg 131->Lys706

### Thr148->Cys726

### Leu155->Val733

Thus the PDK1/PRK2 chimera has the mutations Lys76Gln, Ile119Val, Val127Leu, Thr128Met, Arg131Lys, Thr148Cys and Leu155Val in its PIF binding pocket (wherein the numbering refers to the full length hPDK1 sequence of SEQ ID NO:2). Particularly preferred is a PDK1/PRK2 chimera that has a sequence comprising amino acid residues 24 to 334 of SEQ ID NO:23.

Concerning the chimeric PDK1 that mimics the PIF pocket of hSGK1 (SEQ ID NO:25) the differences between hPDK1 and hSGK1 are shown in FIG. 1E. Eight mutations were introduced to produce the PDK1/SGK1 chimera (first column: PDK1 numbering; second column: SGK1 numbering):

### Lys76->His92

### Arg116->Lys132

### Ile119->Leu135

### Val124->Glu140

### Pro125->Lys141

### Val127->Ile143

### Thr128->Met144

### Thr148->Ser165

Thus the PDK1/SGK1 chimera has the mutations Lys76H is, Arg116Lys, Ile119Leu, Val124Glu, Pro125Lys, Val127Ile, Thr128Met and Thr148Ser in its PIF binding pocket (wherein the numbering refers to the full length hPDK1 sequence of SEQ ID NO:2). Particularly preferred is a PDK1/SGK1chimera that has a sequence comprising amino acid residues 24 to 334 of SEQ ID NO:28.

Concerning the chimeric PDK1 that mimics the PIF pocket of hS6K1 (SEQ ID NO:30) the differences between hPDK1 and hS6K1 are shown in FIG. 1F. Six mutations were introduced to produce the PDK1/S6K1 chimera (first column: PDK1 numbering; second column: S6K1 numbering):

### Ile119->Val131

### Val124->Thr137

### Val127->Thr140

### Thr128->Lys141

### Thr148->Ala161

### Phe157->Leu170

Thus the PDK1/S6K1 chimera has the mutations Ile119Val, Val124Thr, Val127Thr, Thr128Lys, Thr148Ala and Phe157Leu in its PIF binding pocket (wherein the numbering refers to the full length hPDK1 sequence of SEQ ID NO:2). Particularly preferred is a PDK1/S6K1 chimera that has a sequence comprising amino acid residues 24 to 334 of SEQ ID NO:33.

Concerning the chimeric PDK1 that mimics the PIF pocket of hAKT1 (SEQ ID NO:35) the differences between hPDK1 and hAKT1 are shown in FIG. 1G. Eight mutations were introduced to produce the PDK1/AKT1 chimera (first column: PDK1 numbering; second column: AKT1 numbering):

### Lys76->Arg144

### Arg116->Glu184

### Ile119->Val187

### Val127->Thr195

### Thr128->Leu196

### Arg 131->Asn 199

### Ser135->Gln203

### Thr148->Ser216

Thus the PDK1/AKT1 chimera has the mutations Lys76Arg, Arg116Glu, Ile119Val, Val127Thr, Thr128Leu, Arg131Asn, Ser135Gln and Thr148Ser in its PIF binding pocket (wherein the numbering refers to the full length hPDK1 sequence of SEQ ID NO:2). Particularly preferred is a PDK1/AKT1 chimera that has a sequence comprising amino acid residues 24 to 334 of SEQ ID NO:38.

Concerning the chimeric PDK1 that mimics the PIF pocket of hAKT2 (SEQ ID NO:40) the differences between hPDK1 and hAKT2 are shown in FIG. 1H. Six mutations were introduced to produce the PDK1/AKT2 chimera (first column: PDK1 numbering; second column: AKT2 numbering):

### Arg116->Glu186

### Val127->Thr197

### Thr128->Val198

### Arg 131->Ser201

### Ser135->Gln205

### Thr148->Ala218

Thus the PDK1/AKT2 chimera has the mutations Arg116Glu, Val127Thr, Thr128Val, Arg131Ser, Ser135Gln and Thr148Ala in its PIF binding pocket (wherein the numbering refers to the full length hPDK1 sequence of SEQ ID NO:2). Particularly preferred is a PDK1/AKT2 chimera that has a sequence comprising amino acid residues 24 to 334 of SEQ ID NO:43.

Concerning the chimeric PDK1 that mimics the PIF pocket of hRSK2 (SEQ ID NO:45) the differences between hPDK1 and hRSK2 are shown in FIG. 1I. Seven mutations were introduced to produce the PDK1/RSK2 chimera (first column: PDK1 numbering; second column: RSK2 numbering):

### Ile118->Thr106

### Ile119->Leu107

### Val124->Arg112

### Val127->Thr115

### Thr128->Lys116

### Thr148->Ala136

### Phe157->Leu145

Thus the PDK1/RSK2 chimera has the mutations Ile118Thr, Ile119Leu, Val124Arg, Val127Thr, Thr128Lys, Thr148Ala and Phe157Leu in its PIF binding pocket (wherein the numbering refers to the full length hPDK1 sequence of SEQ ID NO:2). Particularly preferred is a PDK1/RSK2 chimera that has a sequence comprising amino acid residues 24 to 334 of SEQ ID NO:48.

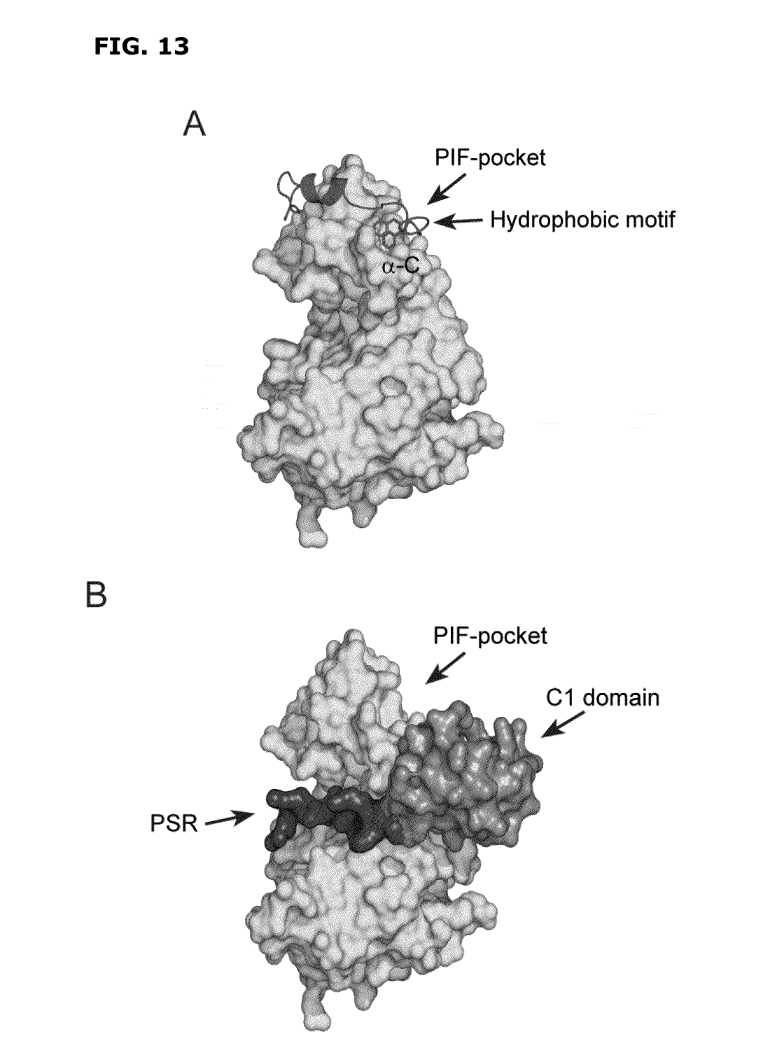

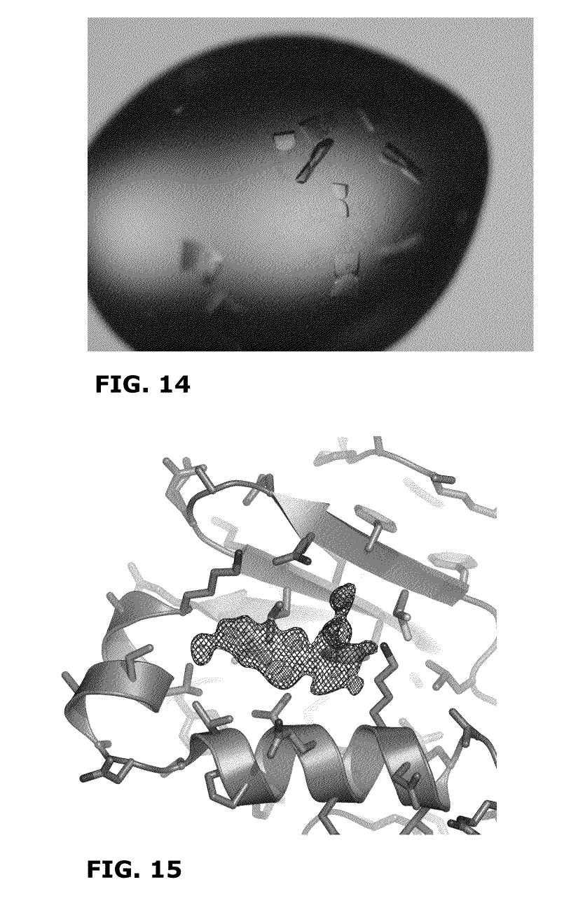

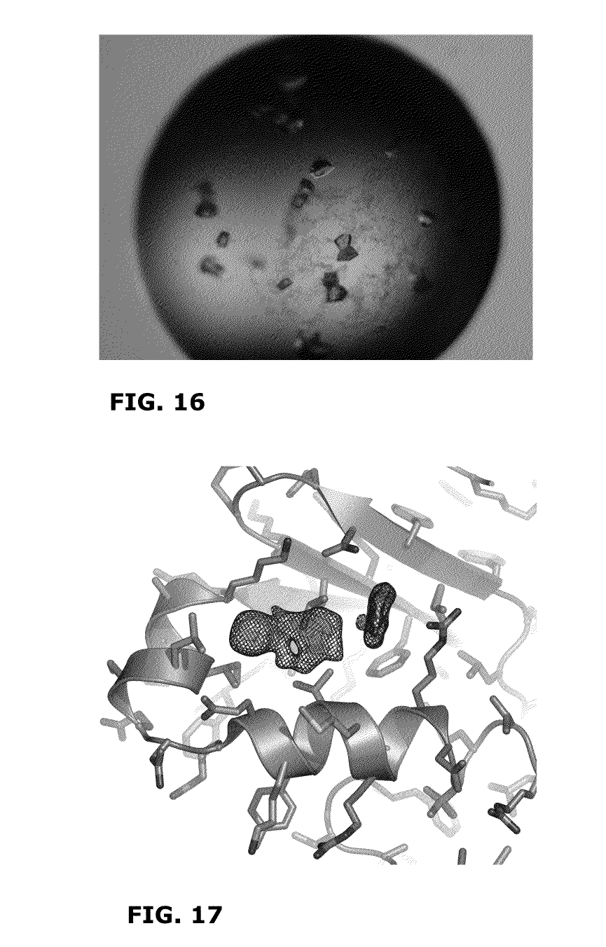

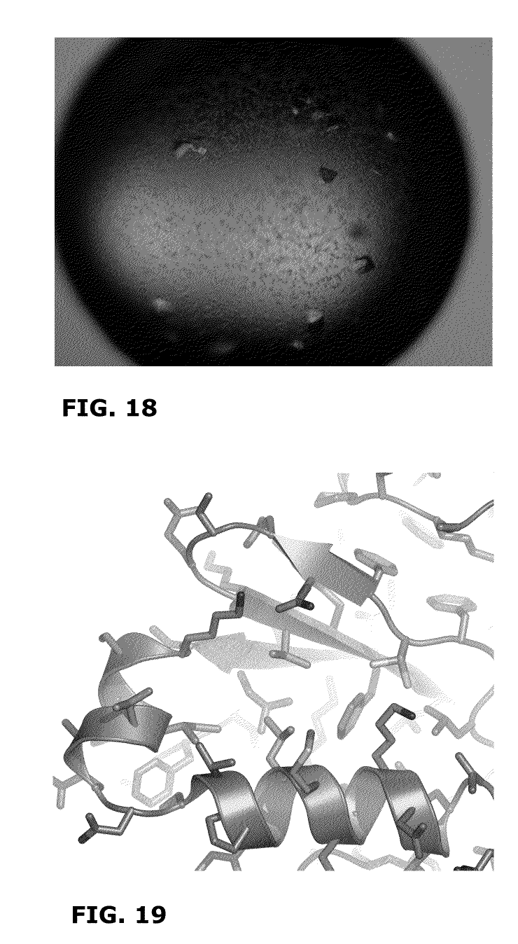

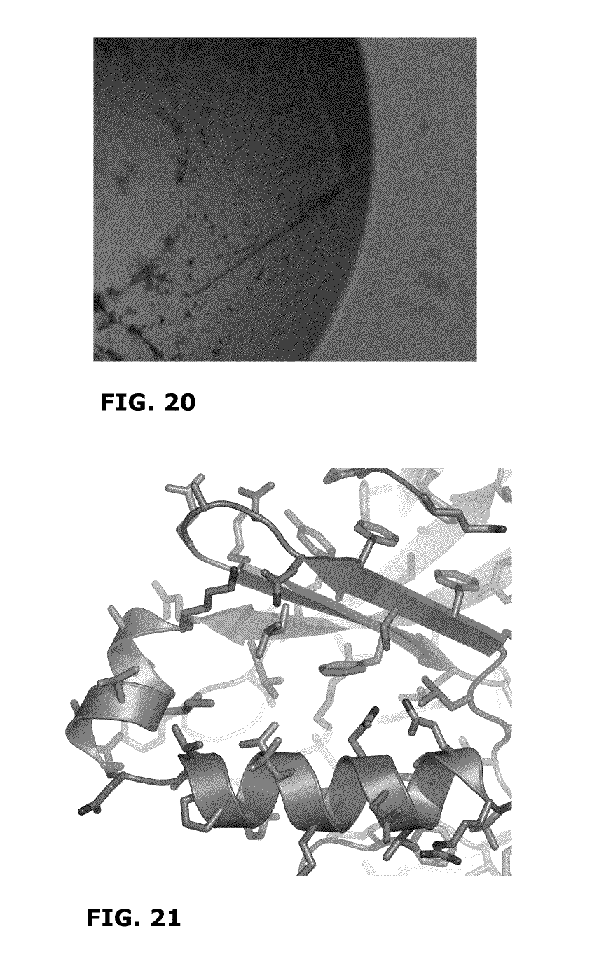

Concerning the chimeric PDK1 that mimics the PIF pocket of hMSK1 (SEQ ID NO:50) the differences between hPDK1 and hMSK1 are shown in FIG. 13. Seven mutations were introduced to produce the PDK1/MSK1 chimera (first column: PDK1 numbering; second column: MSK1 numbering):

### Ile119->Val89

### Val124->Thr95

### Pro125->Glu96

### Val127->Thr98

### Thr128->Arg99

### Thr148->Ala120

### Phe157->Leu129

Thus the PDK1/MSK1 chimera has the mutations Ile119Val, Val124Thr, Pro125Glu, Val127Thr, Thr128Arg, Thr148Ala and Phe157Leu in its PIF binding pocket (wherein the numbering refers to the full length hPDK1 sequence of SEQ ID NO:2). Particularly preferred is a PDK1/MSK1 chimera that has a sequence comprising amino acid residues 24 to 334 of SEQ ID NO:53.

Constructs analogous to the ten PDK1chimera are envisaged to be of universal use in the context of structure-based drug design targeting the PIF-binding pocket of any AGC protein kinase. Following a sequence alignment, differing amino acids at the PIF-binding pocket need to be mutated in order to obtain a PDK1 chimera with a “grafted” pocket of an AGC kinase of choice. Other AGC kinases may be considered in a compound-screening panel, which includes e.g. validated oncology drug targets like PKB/Akt, S6K, RSK or PKCₗ.

In the aspects (1) and (2) above the “derivative” of the chimeric PDK1 may be a C- and/or N-terminal fusion product with a peptide or protein sequence (such as leader and expression sequences, sequences suitable for purification and processing of the mutant protein kinase and other functional protein sequences) and/or with low molecular chemical compound (such as PEG, marker molecules, protective groups). Furthermore it is preferred that the chimeric PDK1 is in a crystalline form.

The method for identifying a compound that binds to the PIF-binding pocket allosteric site mimicked by the chimeric PDK1 protein kinase of aspect (7) of the invention comprises the step of determining the effect of the compound on the chimeric PDK1 of the invention or the ability of the compound to bind to said mutated protein kinase.

It is preferred that the method further comprises (i) the step of determining the effect of the compound on the second protein kinase as defined above or the ability of the compound to bind to said second protein kinase. It is also preferred that the method comprises adding a compound binding to the phosphate binding pocket.

The above aspects (1) to (9) of the invention are based on the unexpected finding that the C1 domain allosterically inhibits the activity of PKCs such as PKCzeta. Indeed the experts in the field did not consider the possibility that there was

an allosteric mechanism of inhibition of PKCzeta mediated by the C1 domain. Moreover, for the whole PKC family it was considered that the PIF-pocket did not serve for the regulation of the kinase activity (Leonard, T. A. et al., 2011, Cell 144, 55-66).

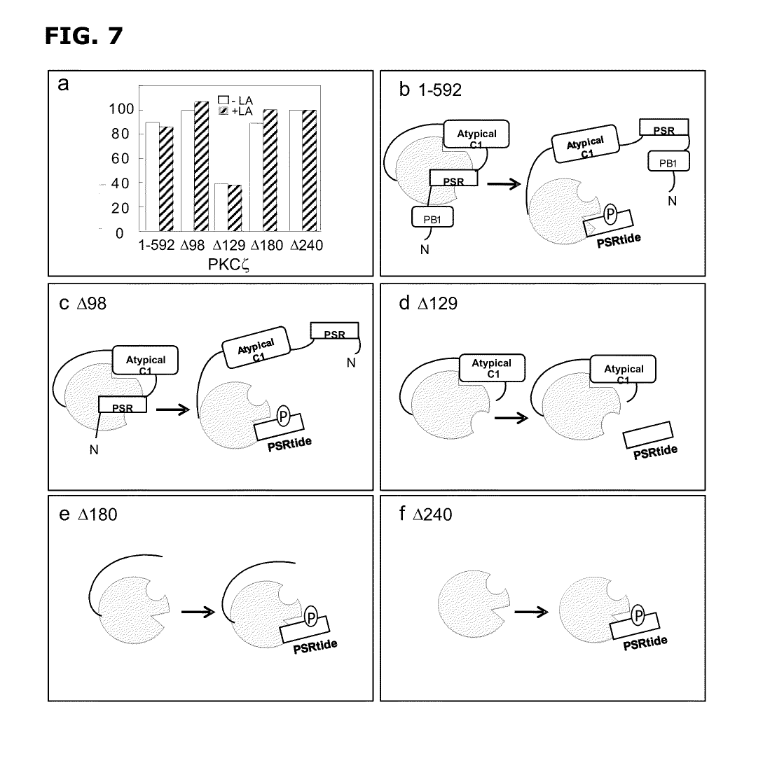

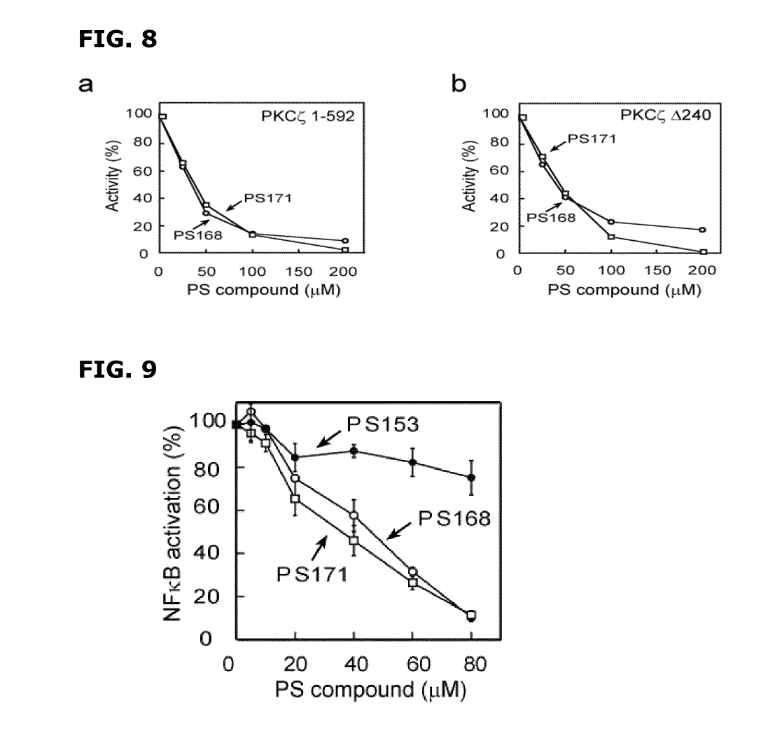

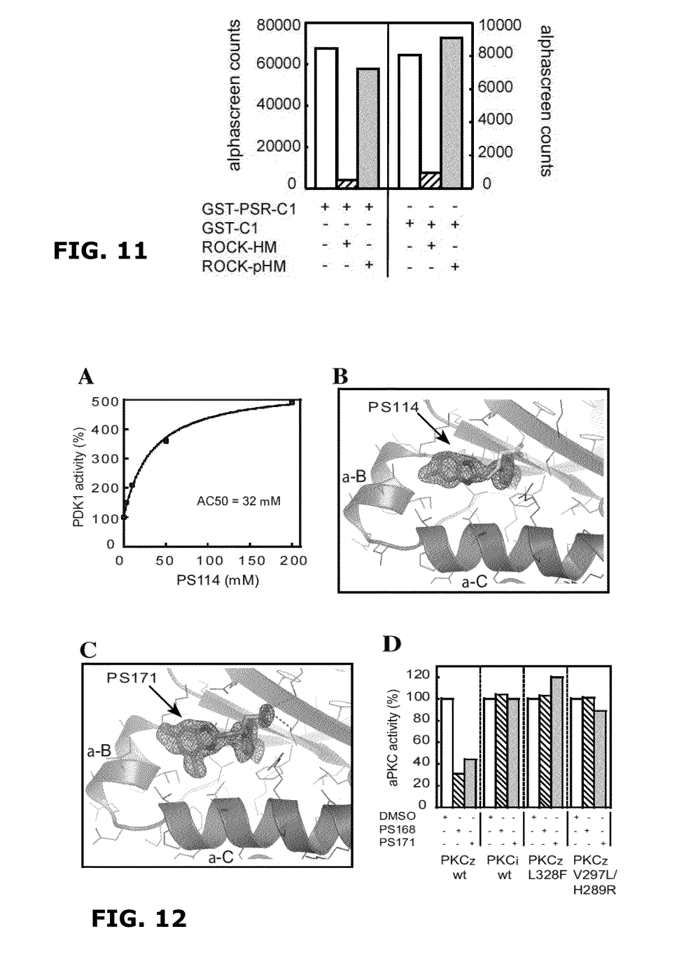

It was now also found that the C1 domain has a direct role on the inhibition and on the process of activation of atypical PKCs. Thus, the C1 domain acts together with the PSR both for the inhibition and for the activation of atypical PKCs. Importantly, all PKC isoforms have in common the pseudosubstrate region (PSR) directly connected to a C1 domain, suggesting that the mechanism is conserved in all the PKC family. In the model shown in FIG. 7 it is shown that the C1 domain directly interacts with the catalytic domain of PKC. Importantly, FIG. 11 provides for experimental data representing formal proof that the C1 domain directly interacts with the catalytic domain. This finding allows the unexpected possibility to screen for compounds that displace the interaction between the C1 domain and the catalytic domain, in order to identify compounds that bind to the PIF-pocket. When the conditions in the alphascreen assay are chosen properly, the assay can identify both inhibitors of the interaction or enhancers of the interaction. Thus, the assay, as described in FIG. 11 also allows to identify compounds that enhance the interaction between the C1 domain and the catalytic domain; Since the interaction inhibits the catalytic activity of PKC, enhancing the interaction will stabilize the inhibited conformation of the PKC isoforms.

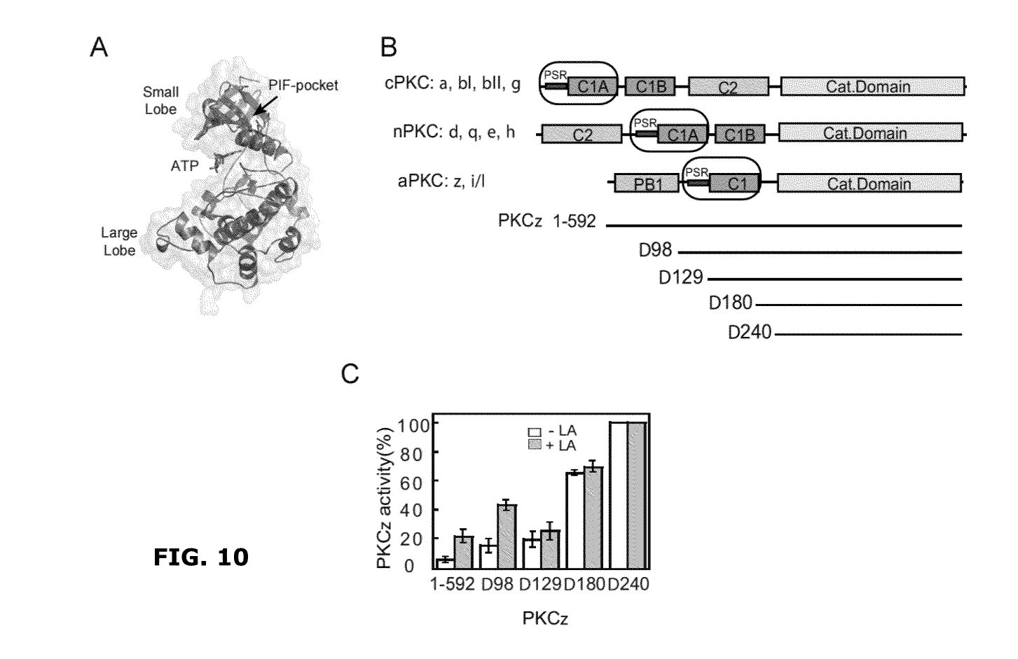

In FIG. 7 it is shown that the N-terminal region of PKCz allosterically inhibits the activity of the kinase domain when using PRStide as a substrate. The model suggests that the C1 domain could bind to the catalytic domain and by doing this, allosterically inhibits PKCz, as depicted in FIG. 7b and FIG. 7d. In FIG. 10, the activity of different PKCz constructs is shown and that it is only after deletion of the C1 domain of PKCz (construct D129) that PKCz constructs gain a major increase in activity. The result confirms that the C1 domain plays a role in the inhibition of PKCz activity.

FIGS. 7 and 10 provide evidence that the C1 domain could allosterically regulate the activity but did not provide a proof on the mechanism. In FIG. 11 evidence is provided that the C1 domain directly interacts with the catalytic domain of PKCz. A novel assay is set-up to investigate the interaction between the C1 domain constructs fused to GST and the catalytic domain of atypical PKCs containing a 6×His-tag. The assay is based on the alphascreen technology using a donor bead coupled to anti-GST antibodies (to bind the GST-PSR-C1 or GST-C1 constructs derived from PKCₗ) and Ni-NTA acceptor beads that bind to His-PKCₗ Δ223 (catalytic domain). The interaction is measured by the emission of light from the acceptor beads that happens when the two beads are in close proximity. GST-PSR-C1 readily interacted with His-PKCₗ Δ223. Interestingly, PIFtide and the HM polypeptide derived from another AGC kinase, ROCK (ROCK-HM, VGNQLPFIGFTYFRENL, SEQ ID NO:59), but not the phosphorylated peptide derived from the HM of ROCK (ROCK-pHM, VGNQLPFIGFTYFRENL), displaced the interaction (FIG. 11). PIFtide and HM polypeptides are known to interact with the PIF-pocket of AGC kinases. Thus, the assay provides evidence that polypeptides binding to the PIF-pocket displace the interaction. In addition, small compounds that inhibit both atypical PKC isoforms and have been co-crystallized with PDK1-PKCz chimera (e.g. PS315), also displace the interaction between the C1 domain and the catalytic domain. Together, the data highlight the important role of the PIF-pocket in the regulation of PKCs and the need to develop tools to improve the methods for drug development to the PIF-pocket on AGC kinases. Notably, the construct lacking the PSR (GST-C1) had low but measurable affinity for His-PKCₗ Δ223 and this interaction was also displaced by the HM polypeptide from ROCK but not by the phosphorylated HM from ROCK (not shown) or by an unrelated polypeptide (RTWALCGTPEYLAPEIILKK, SEQ ID NO:60) derived from the activation loop of PKA (not shown), indicating that the interaction between the C1 domain and the catalytic domain was highly selective. The results show that the C1 domain of an atypical PKC directly interacts with the catalytic domain and suggest an allosteric communication between the PIF-pocket and the C1 domain interaction site. The C1 domain does not possess the classical Phe-Xaa-Xaa-Phe HM sequence and modeling does not predict that it could occupy the hydrophobic PIF-pocket as the HM. Indeed modeling of the pseudosubstrate into the substrate-binding site, allows the interaction of the C1 domain with the external part of the helix α-C that is a main component of the PIF-pocket (FIG. 13). The invention further provides a method for the screening for compounds that interact with the PIF-pocket on PKC isoforms, PDK1 chimeras or other AGC kinases, as well as kits for said method and compounds identified by said method (aspects (10) to (12) of the invention).

It was previously shown that the PIF-pocket was used physiologically along the molecular mechanism of activation of AGC kinases and that small compounds could mimic this regulatory mechanism and activate protein kinase PDK1. It is now shown that the PIF-pocket is also responsible for the mechanism of inhibition of members of AGC kinases and that the PIF-pocket can be targeted with small compounds for the pharmacological activation or the pharmacological inhibition of AGC kinases (FIG. 12).

The mutagenesis of the PIF-pocket on PDK1 to mimic the PIF-pocket on other AGC kinases rendered chimeric proteins that were able to selectively bind compounds and transduce the conformational change that affected the activity of the AGC kinase target of the compound. This was completely unexpected and opens, on its own, a novel tool for the process of drug development. For example, it allows to evaluate the selectivity of compounds to the PIF-pocket on an AGC kinase. Thus, an inhibitor of a protein kinase could potentially target different sites, some known (like the ATP-binding site) and other which may be unexpected. If the mutagenesis of PDK1 to mimic the PIF-pocket of a second AGC kinase rendered a chimeric protein that is affected by a compound to the second AGC kinase, this would provide evidence that the compound was binding to the PIF-pocket. This is an example of an assay where the presence of the double mutant of PDK1 is not necessary.

Similarly to the finding using the dmPDK1 50-359 (FIG. 2), a GST-PDK1 1-556 (full length, without the dm mutation) lost its ability to be activated by PS168 and was inhibited by PS168 when the PIF-pocket was mutated to mimic the PIF-pocket of PKCz. Thus, non-mutated PDK1 50-359 protein and other constructs of PDK1 lacking the (dm) mutations, when mutated at the PIF-pocket to mimic the PIF-pocket of other AGC kinases, are also expected to transduce the conformational change similarly to the dmPDK1 50-359. Non-mutated forms of PDK1 can therefore be mutated to mimic the PIF-pocket of other AGC kinases and be tested for novel crystallization conditions together with allosteric inhibitors of AGC kinases. The preferred construct for mutagenesis to create the chimeric proteins is dmPDK1 50-359. Also preferred is the use of PDK1 50-359 which can be produced in high quality for crystallization studies or other suitable constructs, for example PDK1 76-359 that corresponds to a sequence of aminoacids observed in the crystals of PDK1 in different crystal packings.

The Invention is further disclosed in the following examples, which are however not limiting the invention.

### EXAMPLES

### Materials and Methods

Human embryonic kidney (HEK) 293 cells (ATCC) were cultured on 10 cm dishes in Dulbecco's modified Eagle's medium containing 10% fetal bovine serum (Gibco) and 1% antibiotic antimycotic (Sigma). The U937 cell line was obtained from the ATCC and cultured in RPMI 1640 (Gibco) containing 10% fetal bovine serum (Gibco) and 1% penicillin-streptomycin (Sigma). Materials for mammalian tissue culture were from Greiner. Polyethyleneimine (PEI) “MAX” was from Polysciences Inc. Molecular biology techniques were performed using standard protocols. DNA constructs used for transient transfection were purified from bacteria using a Qiagen plasmid mega kit according to the manufacturer's protocol. Site-directed mutagenesis was performed using a QuikChange kit (Stratagene) following the instructions provided by the manufacturer. DNA sequences were verified by automatic DNA sequencing (Applied Biosystems 3100 Genetic Analyzer). Complete protease inhibitor cocktail tablets were from Roche. “Glutathione Sepharose 4B” and “Ni Sepharose High Performance” were from GE Healthcare. Protein concentration was estimated using a Coomassie reagent from Perbio. The lipid activation mix (“PKC Lipid Activator”), histone H1 and MBP were from Millipore. Phosphatidylserine (1,2-diacyl-sn-glycero-3-phospho-L-serine) from bovine brain was from Sigma. A phospho-specific antibody that recognizes the phosphorylated activation loop of several AGC kinases (anti-phospho-PRK2) was from Upstate Biotechnology. A phospho-specific antibody that recognizes the phosphorylated Z/turn-motif of PKC isoforms (phospho-T641 PKCβ) was from Abcam. Anti-GST was from Cell Signaling. Secondary antibodies IgG IRDye800CW (anti-mouse and anti-rabbit) were from LiCor and IgG Cy5 conjugated (anti-mouse and anti-rabbit) were from Invitrogen. PKA was from Sigma; PKCα was from Millipore; PKCβ, θ, and δ were from ProQinase. PSRtide (biotin-KSIYRRGSRRWRKLYRA; SEQ ID NO:56), used as peptide substrate of PKCζ, and T308tide (KTFCGTPEYLAPEVRR; SEQ ID NO:57), used as substrate of PDK1, were synthesized by JPT Peptide Technologies GmbH. The insect cell expression system and all insect cell related material were from Invitrogen and were used as recommended by the manufacturer.

Expression and Purification of PKCζ and Other AGC Kinases:

For the expression and purification of protein kinases fused to GST, pEBG-2T derived plasmids were transfected into 8×14.5 cm dishes containing HEK293 cells using the PEI method (125 pg PEI and 12.5 mg plasmid/14.5 cm dish). The cells were lysed after 48 h in a buffer containing 50 mM Tris-HCl pH 7.5, 1 mM EGTA, 1 mM EDTA, 1% (w/v) Triton X-100, 1 mM sodium orthovanadate, 50 μM sodium fluoride, 5 mM sodium pyrophosphate, 0.27 M sucrose, 0.1% β-mercaptoethanol, and 1 tablet of protease inhibitor cocktail per 50 ml of buffer. Lysates were frozen in liquid nitrogen and kept at −80° C. until required. Purification involved incubation of the cleared lysate with glutathione sepharose, followed by 2 washes with 0.5 M NaCl in lysis buffer, 8 washes with a buffer containing 50 mM Tris-HCl, 0.1 mM EGTA and 0.1% β-mercaptoethanol (buffer A), and a last wash with buffer A supplemented with 0.26 M sucrose. Elution was performed with this last buffer containing 20 mM glutathione and the GST-fusion protein was cleared from resin by filtration through a “SigmaPrep” spin column (Sigma). GST-fusion proteins were aliquoted, snap frozen in liquid nitrogen and kept at −80° C. until use. Purity at this stage was above 85% as estimated by SDS-PAGE and staining with Coomassie Brilliant Blue R250. PRK2 was expressed from pEBG-2T-PRK2 (Balendran, A. et al. J Biol Chem 275, 20806-13. (2000)), SGK1 from pEBG-2T-SGK1-ΔN[Ser422Asp], PKBα from pEBG-2T-PKBα[Ser473Asp] (Biondi et al., D. R. Embo J 20, 4380-90. (2001)), PKCₗ from pEBG-2T-PKCₗ and PKCζ from pEBG-2T-PKCζ. PDK1 and S6K1 were expressed in Sf9 insect cells using a baculovirus expression system from pFastBac-PDK1 and pFastBac-56K1-T2[Thr412Glu].

Protein Kinase Activity Assay:

The protein kinase activity assays were performed essentially as previously described (Engel, M. et al. Embo J 25, 5469-80 (2006)). The assays were done in a 96-well format and 4 μl aliquots spotted on P81 phospho-cellulose papers (Whatman) using epMotion 5070 (Eppendorf), washed in 0.01% phosphoric acid, dried, and then exposed and analyzed using PhosphoImager technology (FLA-9000 Starion, Fujifilm). Atypical PKC (aPKC) activity assays were performed in a total volume of 20 μl containing 50 mM Tris-HCl pH 7.5, 0.05 mg/ml BSA, 0.1% (v/v) 2-mercaptoethanol, 10 mM MgCl₂, 100 μM [γ³²P]ATP (5-50 cpm/pmol), 0.003% Brij, 30-50 ng of aPKC, and MBP (10 μM) or PSRtide (100 μM) as the substrate. After 15 min pre-incubation, the kinase reaction was started by addition of 6 μl of an ATP-Mg mix. When required, lipid activator (LA) phosphatidylserine (100 ng) or PKC lipid activator mix (1×) was included in the pre-incubation. Low basal activity and consistent activation of 1-592 PKCζ and 498 PKCζ by LA was obtained when the pre-incubation time was started by addition of the whole mix on the enzyme.

The substrates were T308tide (200 μM) for PDK1, Kemptide (100 μM) for PKA, and Crosstide (100 μM) for SGK, PKB, S6K, and PRK2. The activity assays for PKCα, β, θ, and δ were performed in the presence of PKC lipid activator mix (1×) using 3 μM of histone H1 as substrate.

The activity assays were performed in duplicates or triplicates (in the case of the temperature stability assay) with less than 10% difference between the duplicate pairs. The activity assays shown were repeated at least twice with similar results. Moreover, most of the assays shown were repeated multiple times with enzymes from independent purifications with similar results. Representative experiments are shown.

PKCζ Temperature Stability Assay:

In order to measure the thermal stability of PKCζ, the activity of PKCζ towards MBP in the presence or absence of lipid activator was measured after incubation of the enzyme for 2 min at different temperatures (24° C., 37° C., 42° C., 46° C., and 50° C.) previous the activity assay. The samples were then left on ice for 2 min, and 9 μl aliquots were transferred to different tubes containing 11 μl of a solution giving a final concentration of 50 mM Tris (pH 7.5), 0.2 mg/ml MBP, 0.003% Brij, 10 mM MgCl₂, and 100 μM [γ-³²P]ATP (5-50 cpm/pmol). The reaction was stopped after 30 min by adding 5 μl of 200 mM phosphoric acid. 4 μl of each sample were spotted on P81 phosphocellulose papers (Whatman), washed in 0.01% phosphoric acid, dried, exposed, and analyzed using PhosphoImager technology (FLA-9000 Starion, Fujifilm).

PKCζ-PDK1 Interaction Assay:

The protein-protein interaction experiments shown in FIG. 4 were performed by co-transfection of HEK293 cells in 10 cm Petri dishes, as previously described (Dettori, R. et al. J Biol Chem 284, 30318-27 (2009)), with 5 pg of a pEBG-2T-PKCζ plasmid that codes for GST-PKCζ (wild type or truncated mutants) together with 5 pg of a pCMV5-PDK1 plasmid that codes for myc-tagged PDK1. 48 h post-transfection, the cells were lysed in 0.6 ml of lysis buffer. The lysates were cleared by centrifugation at 13,000×g for 10 min at 4° C., and 0.5 ml of supernatant was incubated for 2 h at 4° C. with 30 μl of glutathione sepharose. The beads were washed twice with lysis buffer containing 0.5 M NaCl, followed by two further washes with buffer A. The beads were resuspended in 30 μl of buffer containing 100 mM Tris/HCl (pH 6.8), 4% (w/v) SDS, 20% (v/v) glycerol and 200 mM dithiothreitol and the duplicates for each condition subjected to SDS-polyacrylamide gel electrophoresis followed by immunoblotting. Analysis and quantification of the interaction were performed with a fluorescence infrared imager system (Fujifilm FLA 9000 Starion). We show duplicates of independent transfections and independent pull-down experiments performed in parallel.

PKCζ-Dependent NFκB Signaling in U937 Cells:

In U937 lymphoma cells, tumor necrosis factor alpha (TNFα) dependent activation of NFκB is dependent on PKCζ activity (Folgueira, L. et al. J Virol 70, 223-31 (1996); Muller, G. et al. Embo J 14, 1961-9 (1995)). U937cells were transiently transfected with a plasmid encoding for luciferase under the control of NFκB response elements (pGL4.32 [luc2P/NF-κB-RE/Hygro], Promega). After serum starvation overnight, the cells were incubated in 96-well plates with the compounds or DMSO (0.25%) for 3 h and stimulated with TNFα (50 ng/ml, PeproTech) for 90 min. Bright-Glo Luciferase Assay reagent (Promega) was added and the luciferase activity measured using the multilabel reader station EnVision (Perkin Elmer).

AlphaScreen Interaction Assay:

The AlphaScreen assay was performed according to the manufacturer's general protocol (Perkin Elmer). Reactions were performed in a 25 μl final volume in white 384-well microtiter plates (Greiner). The reaction buffer contained 50 mM Tris-HCl (pH 7.4), 100 mM NaCl, 1 mM dithiotreitol, 0.01% (v/v) Tween-20 and 0.1% (w/v) BSA. 50 nM His6-tagged PKCₗ Δ223 were mixed with 100 nM GST-C1 (PKCₗ 131-186) or 25 nM GST-PSR-C1 (PKCₗ 100-185) in the absence or presence of unlabeled PIFtide or peptides derived from the HM of ROCK (ROCK-HM, VGNQLPFIGFTYFRENL (SEQ ID NO:59) or ROCK-pHM, VGNQLPFIGFT(P)YFRENL) or the activation loop of PKA (RTWALCGTPEYLAPEIILKK; SEQ ID NO:60). Subsequently, 5 μl of beads solution containing nickel chelate-coated acceptor beads and glutathione-coated donor beads was added to the reaction mix in a final concentration of 40 μg/ml for His-PKCₗ Δ223 and GST-C1 or 20 μg/ml for His-PKCₗ Δ223 and GST-PSR-C1, respectively. Proteins and beads were incubated in the dark for 1 h 30 min at room temperature and the emission of light from the acceptor beads was measured in the EnVision reader (Perkin Elmer) and analyzed using the EnVision manager software.

### Example 1

### Expression, Purification and Crystallization of PDK1

PDK1 50-359[Y288G,Q292A] was expressed, purified, concentrated, crystallized, and soaked with compounds as previously described (Hindie, V. et al. Nature Chemical Biology 5, 758-764 (2009); Biondi, R. M. et al. Embo J 21, 4219-28. (2002)). In brief, PDK1 was expressed in Sf9 insect cells as His-tagged PDK1 50-359[Y288G,Q292A] using baculovirus expression technology (Invitrogen). Using this double mutant protein construct, PDK1 crystallized in crystal packing II and diffracted to high resolution.

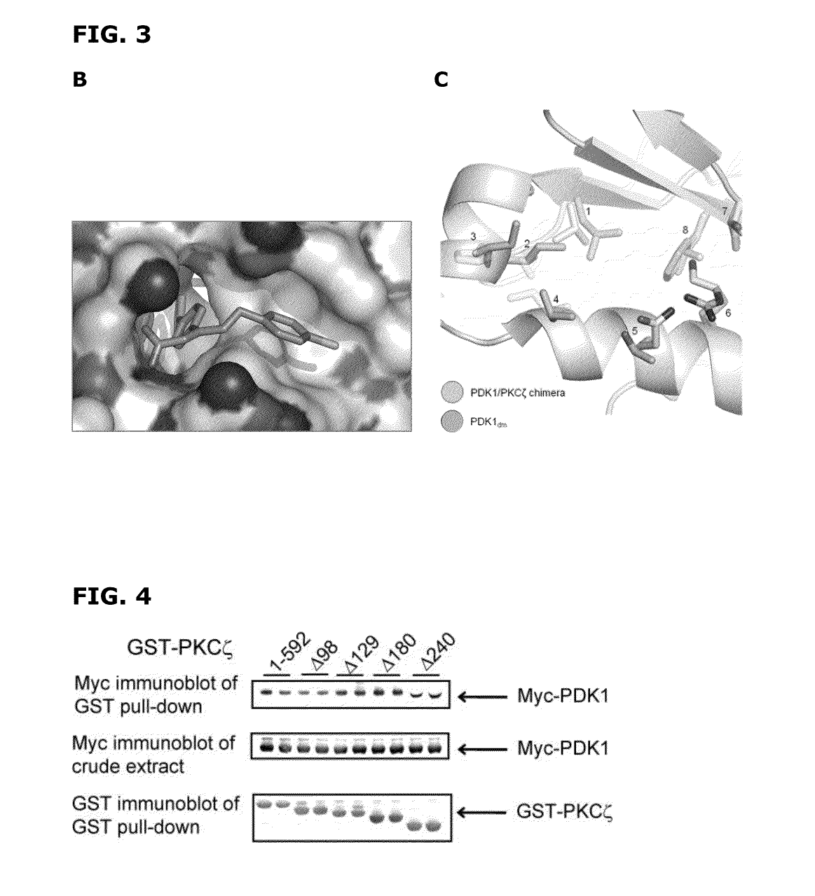

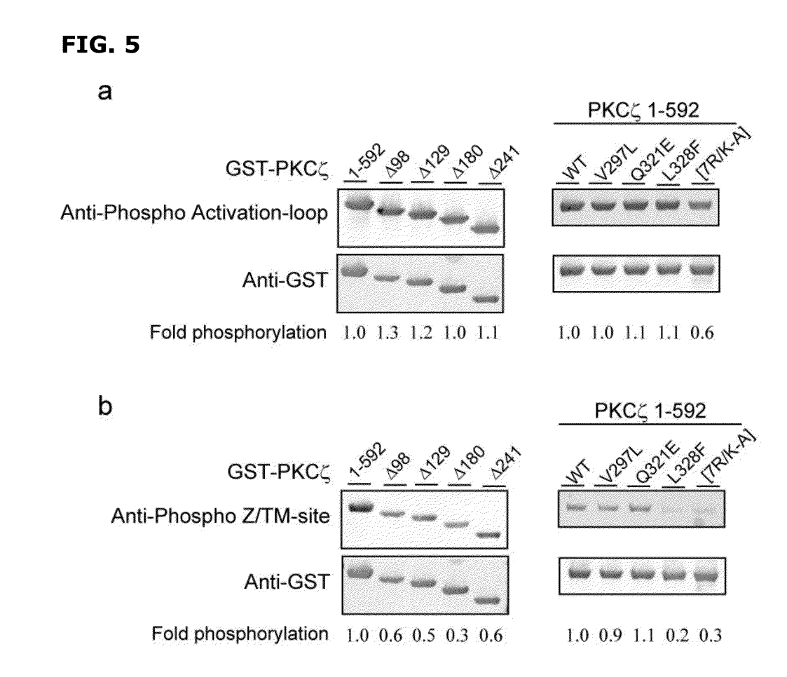

Data collection, structure determination and modeling: X-ray diffraction data were collected at beamline ID23-1 (ESRF, Grenoble) and beamline PXIII (Swiss Light Source, Villigen). Data were processed and scaled using the XDS program package (Kabsch W J Appl. Cryst. 26, 795-800 (1993)). The structure of apo-PDK1 in crystal packing II (Hindie, V. et al. Nature Chemical Biology 5, 758-764 (2009)) (PDB code 3HRC) served as a model for molecular replacement using Phaser (McCoy A. J. et al. J. Appl. Cryst. 40, 658-74 (2007)). PHENIX was used for refinement, including TLS protocols (Adams P. D. et al. Acta Cryst. D66, 213-21 (2010)). Coot was used for manual model building and structural analysis (Emsley P. et al. Acta Cryst. D66 486-501 (2010)). Molecular graphic figures were prepared using PyMOL (DeLano W. L. The PyMOL User's Manual. DeLano Scientific, San Carlos, Calif. (2002)). The statistics for data collection and structure refinement for the PDK1/PKCζ chimera in complexes with compounds PS168 and PS315 are shown in Table III (corresponding to FIGS. 3A and B). A structural model of the PKCζ catalytic domain was created using SWISS-MODEL (Arnold K et al. Bioinformatics 22, 195-201 (2006)) based on the PKCₗ structure 1ZRZ (PDB code) (Messerschmidt A. et al. J. Mol. Biol. 352, 918-31 (2005)).

### Example 2

### Effect of the Pseudosubstrate Region on the Stability of PKCζ

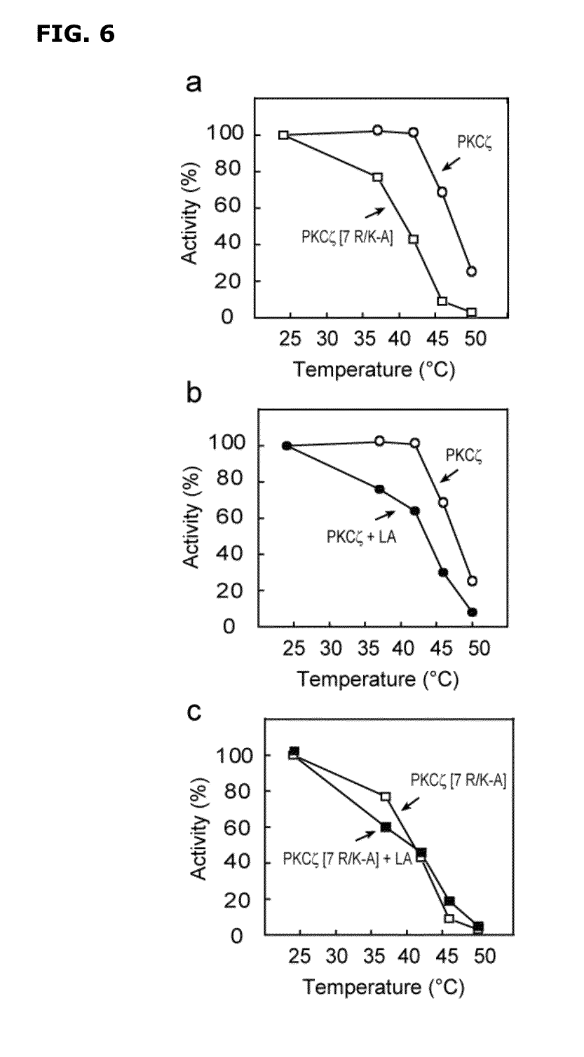

The pseudosubstrate region of PKCs comprises a high number of positively charged residues. To study the role of the pseudosubstrate region on the stability of PKCζ, we prepared a PKCζ construct (PKCζ [7Arg/Lys-Ala]) that had Arg116, Arg117, Arg120, Arg121, Arg123, Lys124 and Arg127 residues within the pseudosubstrate region mutated to Ala (KSIYRRGARRWRKLYRAN; mutated residues underlined (SEQ ID NO:57)). PKCζ [7Arg/Lys-Ala] was significantly less stable to a 2 min temperature shift than the wild type protein (FIG. 6a). The stability data indicated that the positively charged residues within the pseudosubstrate region interacted with other regions of the protein, providing stability. Interestingly, the wild type protein was also less stable in the temperature shift assay when the incubation was performed in the presence of lipid activators (FIG. 6b), suggesting that the binding of lipids to the wild type protein reduced interactions that both inhibited and stabilized the protein. Such loss of stability may be due to loss of interactions involving the different N-terminal regulatory regions of PKCζ (PB1 domain, pseudosubstrate or C1 domain). However, in parallel experiments, PKCζ [7Arg/Lys-Ala] did not further loose protein stability in the presence of lipids (FIG. 6c) indicating that the pseudosubstrate region and not the PB1 domain and the C1 domain mediated the LA-dependent loss of thermal stability. Together, the data suggested that specific interactions mediated by the positively charged residues within the pseudosubstrate segment were responsible for the decreased stability in the presence of lipid activators.

### Example 3

### Effect of PSRtide on the Activity of PKCα

The specific activity of wild type and N-terminally truncated mutants of PKCζ was studied using as a substrate a polypeptide corresponding to the pseudosubstrate region of PKCζ, where Ala 119 is replaced by Ser (PSRtide). In contrast to MBP, this substrate is derived from a region of PKCζ that, to inhibit PKCζ, may prompt direct or indirect specific interactions with its catalytic core. The full length PKCζ protein phosphorylated PSRtide very efficiently, with a specific activity of 60-80 nmol/mg*min (FIG. 7a), supporting the idea that the pseudosubstrate region could indeed bind to the active site on PKCζ. Notably, the LA did not increase the activity of PKCζ towards this substrate, suggesting that the binding of PSRtide to PKCζ overcame its mechanism of inhibition. Moreover, in sharp contrast to the results using MBP as the substrate of the reaction, the truncated mutants did not have increased activity in comparison to full-length PKCζ when PSRtide was used (FIGS. 7a, b, c, e and f). However, the GST-PKCζ [Δ129] construct, which included the C1 domain but not the pseudosubstrate region, had significantly lower specific activity than all other constructs tested (FIGS. 7a and 7d). This result indicated that in GST-PKCζ [Δ129] the inhibitory effect of the C1 domain could not be reversed by PSRtide. PKCζ Δ180 and Δ240, lacking the C1 domain had similar activities as PKCζ 1-592 and Δ98 (FIGS. 7a, e and f). The above data can be explained by the molecular mechanism depicted in the schemes presented in FIGS. 7b, c, d, e and f.

### Example 4

### Effect of PS Compounds on PKCζ-Dependent NFκB Signaling in U937 Cells

U937 lymphoma cells were transiently transfected with a plasmid coding for luciferase under the control of the NFκB promoter. Upon stimulation of the cells with TNFα, an increase in luciferase activity is detected. In U937 cells, the NFκB signaling pathway is dependent on PKCζ activity (Muller, G. et al. Embo J 14, 1961-9 (1995)). PS168 and PS171 inhibited the NFκB signaling in these cells (IC50=50 μM). In contrast, the inactive analogue compound, PS153, did not affect the activation of the NFκB signaling pathway. Together with the in vitro data, this result suggested that PS168 and PS171 are able to bind to the PIF-pocket of PKCζ and inhibit its activity in a cellular environment.

### Example 5

### Determining the Selectivity Profile of Low-Molecular-Weight Compounds PS168, PS171, and PS153 Towards Different AGC Kinases

The results are summarized in Table I. I(a) shows the effect of the compounds on the activity of PKCζ and representatives of other sub-families of related AGC kinases. I(b) shows the effect of the compounds on the activity of PKCζ and other PKC isoforms. Crystal structure has confirmed that the effect of PS171 (50 μM) on the activity of PDK1 is specific, due to the binding of the compound to the PIF-binding pocket. The values indicate the percentage of catalytic activity compared to the activity in the presence of equivalent amounts of DMSO. PKCζ, PRK2, SGK and PKBα [Ser473Asp] were produced as GST-fusion proteins. PDK1 and S6K1-T2-[Thr412Glu] were produced as His-tagged proteins. PKA was purchased from Sigma; PKCα was from Millipore; PKCβ, θ, and δ were from ProQinase.

TABLE I
a

| | Activity (%) | | | | | | |
|---|---|---|---|---|---|---|---|
| | PKCζ | PRK2 | PDK1 | S6K1 | SGK | PKBα | PKA |
| PS168 | 29 | 103 | 294 | 41 | 93 | 87 | 70 |
| PS171 | 48 | 97 | 246 | 47 | 97 | 94 | 91 |
| PS153 | 102 | 101 | 169 | 91 | 108 | 113 | 133 |

b

| | Activity (%) | | | | | |
|---|---|---|---|---|---|---|
| | Atypical PKCs | | Classical PKCs | | Novel PKCs | |
| | PKCζ | PKCι | PKCα | PKCβ | PKCδ | PKCθ |
| PS168 | 29 | 108 | 154 | 148 | 148 | 130 |
| PS171 | 48 | 106 | 145 | 138 | 170 | 118 |
| PS153 | 100 | 98 | 123 | 97 | 103 | 115 |

### Example 6

### Additional Cristallized Chimeric Proteins

In FIG. 3 it is shown that PDK1 can be mutated to mimic the PIF-pocket of PKCz and be crystallized. In another example, following the description of the application we also achieved the crystallization of the PDK1 chimera comprising the mutations in the PIF-pocket to mimic the other atypical PKC isoform, PKCiota (PKCi) as a protein having amino acid residues 24 to 334 of SEQ ID NO:13. Furthermore, we have also achieved the crystallization of the PDK1 chimeras comprising the PIF-pocket of SGK (as a protein having amino acid residues 24 to 334 of SEQ ID NO:28), PRK2 (as a protein having amino acid residues 24 to 334 of SEQ ID NO:23) and PKB (as an AKT1 protein having amino acid residues 24 to 334 of SEQ ID NO:38), see Tables IIa-d. Together, the data indicate that the method here describes serves for the general crystallization of PDK1 chimeras of protein kinases having a regulatory site located at the position of the PIF-binding pocket on PDK1.

TABLE IIa
Data collection statistics of a PDK1/PKCi
chimera crystal soaked with compound PS267.

| | PDK1/PKCi + ATP + | |
|---|---|---|
| Data collection | compound PS267 | |
| Unit cell dimensions, a, b, c (Å) | 149.1, 44.5, 47.8 | |
| Unit cell angles, α, β, γ (°) | 90, 102.0, 90 | |
| Space group | C2 | |
| Wavelength (Å) | 0.91841 | |
| Number of unique reflections | 56419 | |
| Resolution range (Å) | 47-1.42 | (1.52-1.42) |
| Completeness of data (%) | 97.1 | (96.2) |
| Redundancy | 2.5 | (2.6) |
| R_{sym} (%) | 4.9 | (64.7) |
| <I/σ(I)> | 12.8 | (2.0) |

Values in parentheses refer to shells of highest resolution

TABLE IIb
Data collection statistics of a PDK1/SGK chimera
crystal soaked with compound PS238.

| | PDK1/SGK + ATP + | |
|---|---|---|
| Data collection | compound PS238 | |
| Unit cell dimensions, a, b, c (Å) | 148.2, 44.0, 47.3 | |
| Unit cell angles, α, β, γ (°) | 90, 100.2, 90 | |
| Space group | C2 | |
| Wavelength (Å) | 0.91841 | |
| Number of unique reflections | 121817 | |
| Resolution range (Å) | 43-1.1 | (1.2-1.1) |
| Completeness of data (%) | 99.5 | (99.4) |
| Redundancy | 3.7 | (3.4) |
| R_{sym} (%) | 4.9 | (64.7) |
| <I/σ(I)> | 12.8 | (2.0) |

Values in parentheses refer to shells of highest resolution

TABLE IIc
Data collection statistics of a PDK1/PRK2 chimera crystal.

| Data collection | PDK1/PRK2 + ATP | |
|---|---|---|
| Unit cell dimensions, a, b, c (Å) | 148.4, 44.6, 47.5 | |
| Unit cell angles, α, β, γ (°) | 90, 101.0, 90 | |
| Space group | C2 | |
| Wavelength (Å) | 0.91841 | |
| Number of unique reflections | 40421 | |
| Resolution range (Å) | 73-1.6 | (1.7-1.6) |
| Completeness of data (%) | 99.8 | (99.9) |
| Redundancy | 3.4 | (3.4) |
| R_{sym} (%) | 6.6 | (60.3) |
| <I/σ(I)> | 14.3 | (2.1) |

Values in parentheses refer to shells of highest resolution.

TABLE IId
Data collection statistics of a PDK1/PKBα chimera crystal.

| Data collection | PDK1/PKBα + ATP | |
|---|---|---|
| Unit cell dimensions, a, b, c (Å) | 116.9, 116.9, 48.5 | |
| Unit cell angles, α, β, γ (°) | 90, 90, 120 | |
| Space group | P3(1)21 | |
| Wavelength (Å) | 0.91841 | |
| Number of unique reflections | 8675 | |
| Resolution range (Å) | 51-2.9 | (3.0-2.9) |
| Completeness of data (%) | 99.9 | (100) |
| Redundancy | 8.0 | (8.2) |
| R_{sym} (%) | 27.7 | (75.0) |
| <I/σ(I)> | 9.3 | (3.2) |

Values in parentheses refer to shells of highest resolution

TABLE III

| | PDK1/PKCζ- | | PDK1/PKCζ- | |
|---|---|---|---|---|
| | ATP-PS168 | | ATP-PS315 | |
| Data collection | | | | |
| Unit cell dimensions, | 148.4, 44.6, 47.6 | | 148.6, 44.6, 48.0 | |
| a, b, c (Å) | | | | |
| Space group | C2 | | C2 | |
| Wavelength (Å) | 0.9999 | | 0.9999 | |
| Number of unique | 49,444 | | 26,455 | |
| reflections | | | | |
| Resolution range (Å) | 47-1.50 | (1.60-1.50) | 47-1.85 | (1.95-1.85) |
| Completeness of | 99.3 | (98.0) | 99.7 | (99.3) |
| data (%) | | | | |
| Redundancy | 5.3 | (4.6) | 5.9 | (5.8) |
| R_{sym} (%) | 12.9 | (73.6) | 13.3 | (80.4) |
| <I/σ(I)> | 9.2 | (2.6) | 11.4 | (2.6) |
| Refinement | | | | |
| Maximal resolution (Å) | 1.50 | (1.60-1.50) | 1.85 | (1.90-1.85) |
| Monomers per | 1 | | 1 | |
| asymmetric unit | | | | |
| R-factor (%) | 20.5 | (25.8) | 18.0 | (26.2) |
| R_{free} (%) | 23.6 | (26.4) | 23.9 | (31.6) |

| | | |
|---|---|---|
| Wilson B-factor (Å²) | 13.5 | 12.7 |
| R.m.s.d. bond length (Å) | 0.016 | 0.017 |
| R.m.s.d. bond angles (°) | 1.7 | 1.7 |

Data collection and refinement statistics: Values in parentheses refer to shells of highest resolution.

1. A chimeric 3-phosphoinositide-dependent protein kinase 1 (PDK1) having the PDK1 hydrophobic pocket in the position equivalent to the hydrophobic PIF-binding pocket defined by the residues Lys115, Ile118, Ile119, Val124, Val127 and/or Leu155 of full length human PDK1 shown in SEQ ID NO:2 and having the phosphate binding pocket equivalent to the phosphate binding pocket defined by the residues Lys76, Arg131, Thr148 and/or Gln150 of full length hPDK1 shown in SEQ ID NO:2, wherein said mutant protein kinase has a at least two mutations in one of its motives equivalent to AGNEYLIFQK (SEQ ID NO:54) and LDHPFFVK (SEQ ID NO:55) of hPDK1, or a fragment or derivate thereof and wherein the PIF-binding pocket has mutations to mimic a second protein kinase.

2. The chimeric PDK1 of claim 1, which is a mammalian protein kinase derived from the hPDK1 having SEQ ID NO:2.

3. The chimeric PDK1 of claim 1 wherein
(i) the mutation in the motif of SEQ ID NO:54 is a non-conservative mutation, and/or is a mutation of the residues Y or Q; and/or
(ii) the mutation in the motif of SEQ ID NO:55 is a non-conservative mutation, and/or is a mutation of the residues D, H, P, or K.

4. The chimeric PDK1 of claim 1, which
(i) is derived from hPDK1 shown in SEQ ID NO:2 and has at least two mutations at a position corresponding to positions Tyr288 and Gln292, and may have one or more further point mutations at positions corresponding to Lys296 and Ile295, wherein the numbering refers to the full length hPDK1 shown in SEQ ID NO:2; and/or
(ii) is a fragment of the chimeric PDK1 protein kinase that is C- and/or N-terminally truncated and comprises the hydrophobic PIF-binding pocket, the phosphate binding pocket and the motives of SEQ ID NOs:54 and 55.

5. The chimeric PDK1 of claim 1, wherein the second protein kinase that is mimicked by the PIF pocket of the chimeric PDK1 is a mammalian protein kinase grouped within the AGC group of protein kinases, or is a protein kinase from an infectious organism.

6. The chimeric PDK1 of claim 5, wherein the second protein kinase that is mimicked by the PIF pocket of the chimeric PDK1 is
(i) hPKCζ (SEQ ID NO:5) and the chimeric PDK1 protein kinase has the mutations Leu113Val, Ile118Val, Ile119H is, Val124Ile, Thr128Gln, Arg131Lys, Thr148Cys and Phe157Leu in its PIF binding pocket (wherein the numbering refers to the full length hPDK1 sequence of SEQ ID NO:2);
(ii) hPKCₗ (SEQ ID NO:10) and the chimeric PDK1 protein kinase has the mutations Lys76Ser, Leu113Val, Ile118Val, Ile119Asn, Val124Ile, Thr128Gln, Arg131Lys and Thr148Cys in its PIF binding pocket (wherein the numbering refers to the full length hPDK1 sequence of SEQ ID NO:2);
(iii) Candida albicans PKH1 (SEQ ID NO:15) and the chimeric PDK1 protein kinase has the mutations Lys76Arg, Leu128Asn286 and Arg131Lys in its PIF binding pocket (wherein the numbering refers to the full length hPDK1 sequence of SEQ ID NO:2), preferably has a sequence comprising amino acid residues 24 to 334 of SEQ ID NO:18;
(iv) hPRK2 (SEQ ID NO:20) and the chimeric PDK1 protein kinase has the mutations Lys76Gln, Ile119Val, Val127Leu, Thr128Met, Arg131Lys, Thr148Cys and Leu155Val in its PIF binding pocket (wherein the numbering refers to the full length hPDK1 sequence of SEQ ID NO:2);
(v) hSGK1 (SEQ ID NO:25) and the chimeric PDK1 protein kinase has the mutations Lys76H is, Arg116Lys, Ile119Leu, Val124Glu, Pro125Lys, Val127Ile, Thr128Met and Thr148Ser in its PIF binding pocket (wherein the numbering refers to the full length hPDK1 sequence of SEQ ID NO:2);
(vi) hS6K1 (SEQ ID NO:30) and the chimeric PDK1 protein kinase has the mutations Ile119Val, Val124Thr, Val127Thr, Thr128Lys, Thr148Ala and Phe157Leu in its PIF binding pocket (wherein the numbering refers to the full length hPDK1 sequence of SEQ ID NO:2);
(vii) hAKT1 (SEQ ID NO:35) and the chimeric PDK1 protein kinase has the mutations Lys76Arg, Arg116Glu, Ile119Val, Val127Thr, Thr128Leu, Arg131Asn, Ser135Gln and Thr148Ser in its PIF binding pocket (wherein the numbering refers to the full length hPDK1 sequence of SEQ ID NO:2);
(viii) hAKT2 (SEQ ID NO:40) and the chimeric PDK1 protein kinase has the mutations Arg116Glu, Val127Thr, Thr128Val, Arg131Ser, Ser135Gln and Thr148Ala in its PIF binding pocket (wherein the numbering refers to the full length hPDK1 sequence of SEQ ID NO:2);
(ix) hRSK2 (SEQ ID NO:45) and the chimeric PDK1 protein kinase has the mutations Ile118Thr, Ile119Leu, Val124Arg, Val127Thr, Thr128Lys, Thr148Ala and Phe157Leu in its PIF binding pocket (wherein the numbering refers to the full length hPDK1 sequence of SEQ ID NO:2); or
(x) hMSK1 (SEQ ID NO:50) and the chimeric PDK1 protein kinase has the mutations Ile119Val, Val124Thr, Pro125Glu, Val127Thr, Thr128Arg, Thr148Ala and Phe157Leu in its PIF binding pocket (wherein the numbering refers to the full length hPDK1 sequence of SEQ ID NO:2).

7. The chimeric PDK1 of claim 1, wherein
(i) the derivative of the chimeric PDK1 is a C- and/or N-terminal fusion product with a peptide or protein sequence and/or with a low molecular chemical compound; and/or
(ii) the chimeric PDK1 is in a crystalline form.

8. A polynucleotide sequence encoding the chimeric PDK1 of claim 1.

9. A vector comprising the polynucleotide sequence of claim 8.

10. A host cell comprising the polynucleotide sequence of claim 8.

11. A process for producing a chimeric PDK1, said process comprising culturing the host cell of claim 10 and isolating said chimeric PDK1.

12. A method for identifying a compound that binds to a PIF-binding pocket allosteric site mimicked by a chimeric PDK1 protein kinase, said method comprising the step of determining the effect of the compound on the chimeric PDK1 of claim 1 or the ability of the compound to bind to said chimeric PDK1 protein kinase.

13. The method of claim 12, which further comprises
(i) the step of determining the effect of the compound on a second protein kinase or the ability of the compound to bind to said second protein kinase; and/or
(ii) adding a compound binding to the phosphate binding pocket.

14. A kit for use in identifying a compound that binds to a PIF-binding pocket allosteric site mimicked by a chimeric PDK1 protein kinase, said kit comprising a chimeric PDK1 of claim 1.

15. A compound identified by the method of claim 12 binding to the PIF-binding pocket allosteric site of the chimeric PDK1.

16. A method for screening for a compound that interacts with the PIF-pocket of an AGC kinases, which method comprises the step of determining the effect of the compound to be tested on the interaction between a first protein comprising the PIF-pocket of said AGC kinase and a second protein comprising the C1-domain of same or different AGC kinase.

17. A method for screening for a compound that interacts with the PIF-pocket of an AGC kinases, which method comprises the step of determining the effect of the compound to be tested on the interaction between a first protein comprising the PIF-pocket of said AGC kinase and a second protein comprising the C1-domain of same or different AGC kinase, wherein
(i) the AGC kinase is a PKC isoform, a PDK1 chimera, notably a chimeric PDK1 as defined in claim 1, or other AGC kinase; and/or
(ii) the C1-domain of the second protein is from the same AGC kinase as the PIF-pocket of the first protein; and/or
(iii) the method is performed by an AlphaScreen assay protocol, where the first and second proteins are attached to donor and acceptor beads and the interaction is determined by detection of the emission of light from the donor beads.

18. A kit for performing the method of claim 16, which comprises first and second proteins as defined in claim 16.

19. A compound identified by the method of claim 16, binding to the PIF-binding pocket of an AGC kinase.


SEQUENCE LISTING (60 sequences)

SEQ ID NO: 1 - DNA, 1668 bp, Homo sapiens
  CDS (1)..(1668)
atggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagattgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtttgtagaaaataatttaatactaaagatgggcccagtggataagcggaagggtttatttgcaagacgacgacagctgttgctcacagaaggaccacatttatattatgtggatcctgtcaacaaagttctgaaaggtgaaattccttggtcacaagaacttcgaccagaggccaagaattttaaaactttctttgtccacacgcctaacaggacgtattatctgatggaccccagcgggaacgcacacaagtggtgcaggaagatccaggaggtttggaggcagcgataccagagccacccggacgccgctgtgcag

SEQ ID NO: 2 - protein, 556 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 3 - protein, 312 aa, Artificial
  hPDK1 mutant
GAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLTA

SEQ ID NO: 4 - DNA, 1776 bp, Homo sapiens
  CDS (1)..(1776)
atgcccagcaggaccggccccaagatggaagggagcggcggccgcgtccgcctcaaggcgcattacgggggggacatcttcatcaccagcgtggacgccgccacgaccttcgaggagctctgtgaggaagtgagagacatgtgtcgtctgcaccagcagcacccgctcaccctcaagtgggtggacagcgaaggtgacccttgcacggtgtcctcccagatggagctggaagaggctttccgcctggcccgtcagtgcagggatgaaggcctcatcattcatgttttcccgagcacccctgagcagcctggcctgccatgtccgggagaagacaaatctatctaccgccggggagccagaagatggaggaagctgtaccgtgccaacggccacctcttccaagccaagcgctttaacaggagagcgtactgcggtcagtgcagcgagaggatatggggcctcgcgaggcaaggctacaggtgcatcaactgcaaactgctggtccataagcgctgccacggcctcgtcccgctgacctgcaggaagcatatggattctgtcatgccttcccaagagcctccagtagacgacaagaacgaggacgccgaccttccttccgaggagacagatggaattgcttacatttcctcatcccggaagcatgacagcattaaagacgactcggaggaccttaagccagttatcgatgggatggatggaatcaaaatctctcaggggcttgggctgcaggactttgacctaatcagagtcatcgggcgcgggagctacgccaaggttctcctggtgcggttgaagaagaatgaccaaatttacgccatgaaagtggtgaagaaagagctggtgcatgatgacgaggatattgactgggtacagacagagaagcacgtgtttgagcaggcatccagcaaccccttcctggtcggattacactcctgcttccagacgacaagtcggttgttcctggtcattgagtacgtcaacggcggggacctgatgttccacatgcagaggcagaggaagctccctgaggagcacgccaggttctacgcggccgagatctgcatcgccctcaacttcctgcacgagagggggatcatctacagggacctgaagctggacaacgtcctcctggatgcggacgggcacatcaagctcacagactacggcatgtgcaaggaaggcctgggccctggtgacacaacgagcactttctgcggaaccccgaattacatcgcccccgaaatcctgcggggagaggagtacgggttcagcgtggactggtgggcgctgggagtcctcatgtttgagatgatggccgggcgctccccgttcgacatcatcaccgacaacccggacatgaacacagaggactaccttttccaagtgatcctggagaagcccatccggatcccccggttcctgtccgtcaaagcctcccatgttttaaaaggatttttaaataaggaccccaaagagaggctcggctgccggccacagactggattttctgacatcaagtcccacgcgttcttccgcagcatagactgggacttgctggagaagaagcaggcgctccctccattccagccacagatcacagacgactacggtctggacaactttgacacacagttcaccagcgagcccgtgcagctgaccccagacgatgaggatgccataaagaggatcgaccagtcagagttcgaaggctttgagtatatcaacccattattgctgtccaccgaggagtcggtg

SEQ ID NO: 5 - protein, 592 aa, Homo sapiens
MPSRTGPKMEGSGGRVRLKAHYGGDIFITSVDAATTFEELCEEVRDMCRLHQQHPLTLKWVDSEGDPCTVSSQMELEEAFRLARQCRDEGLIIHVFPSTPEQPGLPCPGEDKSIYRRGARRWRKLYRANGHLFQAKRFNRRAYCGQCSERIWGLARQGYRCINCKLLVHKRCHGLVPLTCRKHMDSVMPSQEPPVDDKNEDADLPSEETDGIAYISSSRKHDSIKDDSEDLKPVIDGMDGIKISQGLGLQDFDLIRVIGRGSYAKVLLVRLKKNDQIYAMKVVKKELVHDDEDIDWVQTEKHVFEQASSNPFLVGLHSCFQTTSRLFLVIEYVNGGDLMFHMQRQRKLPEEHARFYAAEICIALNFLHERGIIYRDLKLDNVLLDADGHIKLTDYGMCKEGLGPGDTTSTFCGTPNYIAPEILRGEEYGFSVDWWALGVLMFEMMAGRSPFDIITDNPDMNTEDYLFQVILEKPIRIPRFLSVKASHVLKGFLNKDPKERLGCRPQTGFSDIKSHAFFRSIDWDLLEKKQALPPFQPQITDDYGLDNFDTQFTSEPVQLTPDDEDAIKRIDQSEFEGFEYINPLLLSTEESV

SEQ ID NO: 6 - protein, 50 aa, Artificial
  PDK1 fragment
IKILEKRHIIKENKVPYVTRERDVMSRLDHPFFVKLYFTFQDDEKLYFGL

SEQ ID NO: 7 - protein, 51 aa, Artificial
  PKCzeta fragment
MKVVKKELVHDDEDIDWVQTEKHVFEQASSNPFLVGLHSCFQTTSRLFLVI

SEQ ID NO: 8 - protein, 334 aa, Artificial
  PDK1-PKCzeta chimera
MSYYHHHHHHDYDIPTTENLYFQGAMDGTAAEPRPGAGSLQHAQPPPQPRKKRPEDFKFGKILGEGSFSTVVLARELATSREYAIKIVEKRHVHKENKIPYVQREKDVMSRLDHPFFVKLYFCFQDDEKLYLGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 9 - DNA, 1761 bp, Homo sapiens
  CDS (1)..(1761)
atgtcccacacggtcgcaggcggcggcagcggggaccattcccaccaggtccgggtgaaagcctactaccgcggggatatcatgataacacattttgaaccttccatctcctttgagggcctttgcaatgaggttcgagacatgtgttcttttgacaacgaacagctcttcaccatgaaatggatagatgaggaaggagacccgtgtacagtatcatctcagttggagttagaagaagcctttagactttatgagctaaacaaggattctgaactcttgattcatgtgttcccttgtgtaccagaacgtcctgggatgccttgtccaggagaagataaatccatctaccgtagaggtgcacgccgctggagaaagctttattgtgccaatggccacactttccaagccaagcgtttcaacaggcgtgctcactgtgccatctgcacagaccgaatatggggacttggacgccaaggatataagtgcatcaactgcaaactcttggttcataagaagtgccataaactcgtcacaattgaatgtgggcggcattctttgccacaggaaccagtgatgcccatggatcagtcatccatgcattctgaccatgcacagacagtaattccatataatccttcaagtcatgagagtttggatcaagttggtgaagaaaaagaggcaatgaacaccagggaaagtggcaaagcttcatccagtctaggtcttcaggattttgatttgctccgggtaataggaagaggaagttatgccaaagtactgttggttcgattaaaaaaaacagatcgtatttatgcaatgaaagttgtgaaaaaagagcttgttaatgatgatgaggatattgattgggtacagacagagaagcatgtgtttgagcaggcatccaatcatcctttccttgttgggctgcattcttgctttcagacagaaagcagattgttctttgttatagagtatgtaaatggaggagacctaatgtttcatatgcagcgacaaagaaaacttcctgaagaacatgccagattttactctgcagaaatcagtctagcattaaattatcttcatgagcgagggataatttatagagatttgaaactggacaatgtattactggactctgaaggccacattaaactcactgactacggcatgtgtaaggaaggattacggccaggagatacaaccagcactttctgtggtactcctaattacattgctcctgaaattttaagaggagaagattatggtttcagtgttgactggtgggctcttggagtgctcatgtttgagatgatggcaggaaggtctccatttgatattgttgggagctccgataaccctgaccagaacacagaggattatctcttccaagttattttggaaaaacaaattcgcataccacgttctctgtctgtaaaagctgcaagtgttctgaagagttttcttaataaggaccctaaggaacgattgggttgtcatcctcaaacaggatttgctgatattcagggacacccgttcttccgaaatgttgattgggatatgatggagcaaaaacaggtggtacctccctttaaaccaaatatttctggggaatttggtttggacaactttgattctcagtttactaatgaacctgtccagctcactccagatgacgatgacattgtgaggaagattgatcagtctgaatttgaaggttttgagtatatcaatcctcttttgatgtctgcagaagaatgtgtc

SEQ ID NO: 10 - protein, 587 aa, Homo sapiens
MSHTVAGGGSGDHSHQVRVKAYYRGDIMITHFEPSISFEGLCNEVRDMCSFDNEQLFTMKWIDEEGDPCTVSSQLELEEAFRLYELNKDSELLIHVFPCVPERPGMPCPGEDKSIYRRGARRWRKLYCANGHTFQAKRFNRRAHCAICTDRIWGLGRQGYKCINCKLLVHKKCHKLVTIECGRHSLPQEPVMPMDQSSMHSDHAQTVIPYNPSSHESLDQVGEEKEAMNTRESGKASSSLGLQDFDLLRVIGRGSYAKVLLVRLKKTDRIYAMKVVKKELVNDDEDIDWVQTEKHVFEQASNHPFLVGLHSCFQTESRLFFVIEYVNGGDLMFHMQRQRKLPEEHARFYSAEISLALNYLHERGIIYRDLKLDNVLLDSEGHIKLTDYGMCKEGLRPGDTTSTFCGTPNYIAPEILRGEDYGFSVDWWALGVLMFEMMAGRSPFDIVGSSDNPDQNTEDYLFQVILEKQIRIPRSLSVKAASVLKSFLNKDPKERLGCHPQTGFADIQGHPFFRNVDWDMMEQKQVVPPFKPNISGEFGLDNFDSQFTNEPVQLTPDDDDIVRKIDQSEFEGFEYINPLLMSAEECV

SEQ ID NO: 11 - protein, 77 aa, Artificial
  PDK1 fragment
PRKKRPEDFKFGKILGEGSFSTVVLARELATSREYAIKILEKRHIIKENKVPYVTRERDVMSRLDHPFFVKLYFTFQ

SEQ ID NO: 12 - protein, 78 aa, Artificial
  PKCiota fragment
SSSLGLQDFDLLRVIGRGSYAKVLLVRLKKTDRIYAMKVVKKELVNDDEDIDWVQTEKHVFEQASNHPFLVGLHSCFQ

SEQ ID NO: 13 - protein, 334 aa, Artificial
  PDK1-PKCiota chimera
MSYYHHHHHHDYDIPTTENLYFQGAMDGTAAEPRPGAGSLQHAQPPPQPRSKRPEDFKFGKILGEGSFSTVVLARELATSREYAIKIVEKRHVNKENKIPYVQREKDVMSRLDHPFFVKLYFCFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 14 - DNA, 2835 bp, Candida albicans
  CDS (1)..(2835)
atgcataaatttagatattctttgcaccaacactatagcaaacgcaattcaagtgacaaatccaaagacagtccaattagccaaaacagcaatgaagaaaatgattcgactaaattaagttcaagtagtcttcaagacttacatgatgatctcgatgatatttataacaactatactttagcacagggtaccaataacaacagtgtagatacattggattctgaaaataatcaagctataaataagtttattgataaacctccagcaattcatggtatggaaccacaactaccggtgatgcacgtttcttcacgattatcttccttaggtaataccaccaatgaaaccggtgaaagcatcgccaaaagtgcaccaggaactccgttatcttcacattcatttgatttcagaccgcatcatcctcgtgcagtaacaaactcatccctcaatgtattgttagacacccctaatgtcagttccgaattcaatcatttagtggatcaaacaccacccaatgagtcggtagaaaggtttgacgacagtaataatactgtggacaatacagaagaggaagaaaataatgatgatacagacgaaataccaaaatccgaaacattgaaacaaaacgaggagaattgggaaaaaaagggtgctgcagttaaaactatcaagactatggatggagaaatgaaaactattcggcgaaatgttactgatttcaaatttggtaaagaattgggtgaaggttcatattccacggtgattttagccactgataagattactggtaaacaatatgctgtaaaagtacttgataagcgacatattataaaagaaaagaaagtcaagtatgtcaatatagaaaaacatgcattgaatcgattaagtaatagattaggggttatttcattatatttcaccttccaggataaagattcgctttattttgttttggattatgcttcaaatggtgaattattgacattgatcaagagatacaatactttaaatgaggaatgtactagacattttggtgcacaaatattagatgctattaaatatatgcatgataatggtgttatacatcgagacctaaaaccagagaatatattattagatgacaaaatgagaattcaaattacagattttggtactgcaagattattagagaaaaagaatgatgaaagtgaagaatacccagtggatgtaagagcaaaatcatttgttggaaccgctgaatatgtatcccctgaattattagaaaataagtattgtggtaaacctggagatgtttgggcttttggttgcatcatatatcaaatgattgctgggaaaccaccatttaaggcaactaatgaatatttaacgtttcaaaaaattacgaaattgcaatttgcgtttagtgcaggattccctacaattattagagatttaataaagaagattcttgtgttgcaaccttcacgacgtgccaccattccagaaatacaaaaacattactttttccaatcggtcgactttaaagattttgatctgatttggttgtctgatcctcctgaaataggaccttataaaatgacagcaaaatccatgatgaaagtaccggaattgaataaggcacctataaccacagtcattaagaagaatgtgaagaaatccacaaactcaaattcaaataccaacaatgtcgccactgctgttggtggtagtagtagtaacggacataaagggtcatcaccgactcctgagaaagagccgagcccagctactattaataacaagtccacagaaaaagttagtgccgctagtgtagctgcatatgttttaaacaaaccagctacaaaccaaaattccagtacatccgaggattcatctaagcgtagcagcaactccaatgaaactcgcaaactttcatattcacaacaggattatattccgggaacaaatattttacgtccacagattagtactagaccgtcagtaggatcttatgtgaaaaccacaccatcaaaggatagaaaaacattaaccaaggtcccactgaatatccatcaacaacaagaaaaagtgaaaccgaaagtaatggaagtgaagccagcaactacattggaagcagcatgggaaccatatttaacccatccagatgaaagaatacttcgtattggtccagttattgctcataaagaaccaacagaaccatttgaaaagaagaataaagcatctttacatatatcacctttggatataaataaagaacaaagaagtagatccaatactagtttacttacacaaattgtaaatgaagtaaacaataacaccagcgaattgaaaaaagtggaaaatgctgatgaatcacttgccattattgaaccacaatataatatgaagagaagtccaacttctgatagtaagaaaagtatggatattgaaagatctgcatctacttctggaagtagaattagtaagaaggcaattttcaaaaaattggggtttagtcatttagaaaaaaatgatagtgaagaatcaaatggtcctagtttaacggaaaaaccacaaacttgtacattggttgttacaactcatggtcgagcattacttttcattagaaatgatatagaatccaattatcttttaattgctgaaatcaaattgaaatatccatttattcatttccaagaattagttatatcacaaactaaattttctaaattagtaccatcagtcggagtatttgtcattagttcaattgataattcattaatttttgaagtagaaaaatttgaagtgaatcaatggactgaagcattagctaaatctaaatataatgaaatataa

SEQ ID NO: 15 - protein, 944 aa, Candida albicans
MHKFRYSLHQHYSKRNSSDKSKDSPISQNSNEENDSTKLSSSSLQDLHDDLDDIYNNYTLAQGTNNNSVDTLDSENNQAINKFIDKPPAIHGMEPQLPVMHVSSRLSSLGNTTNETGESIAKSAPGTPLSSHSFDFRPHHPRAVTNSSLNVLLDTPNVSSEFNHLVDQTPPNESVERFDDSNNTVDNTEEEENNDDTDEIPKSETLKQNEENWEKKGAAVKTIKTMDGEMKTIRRNVTDFKFGKELGEGSYSTVILATDKITGKQYAVKVLDKRHIIKEKKVKYVNIEKHALNRLSNRLGVISLYFTFQDKDSLYFVLDYASNGELLTLIKRYNTLNEECTRHFGAQILDAIKYMHDNGVIHRDLKPENILLDDKMRIQITDFGTARLLEKKNDESEEYPVDVRAKSFVGTAEYVSPELLENKYCGKPGDVWAFGCIIYQMIAGKPPFKATNEYLTFQKITKLQFAFSAGFPTIIRDLIKKILVLQPSRRATIPEIQKHYFFQSVDFKDFDLIWLSDPPEIGPYKMTAKSMMKVPELNKAPITTVIKKNVKKSTNSNSNTNNVATAVGGSSSNGHKGSSPTPEKEPSPATINNKSTEKVSAASVAAYVLNKPATNQNSSTSEDSSKRSSNSNETRKLSYSQQDYIPGTNILRPQISTRPSVGSYVKTTPSKDRKTLTKVPLNIHQQQEKVKPKVMEVKPATTLEAAWEPYLTHPDERILRIGPVIAHKEPTEPFEKKNKASLHISPLDINKEQRSRSNTSLLTQIVNEVNNNTSELKKVENADESLAIIEPQYNMKRSPTSDSKKSMDIERSASTSGSRISKKAIFKKLGFSHLEKNDSEESNGPSLTEKPQTCTLVVTTHGRALLFIRNDIESNYLLIAEIKLKYPFIHFQELVISQTKFSKLVPSVGVFVISSIDNSLIFEVEKFEVNQWTEALAKSKYNEI

SEQ ID NO: 16 - protein, 60 aa, Artificial
  PDK1 fragment
PRKKRPEDFKFGKILGEGSFSTVVLARELATSREYAIKILEKRHIIKENKVPYVTRERDV

SEQ ID NO: 17 - protein, 60 aa, Artificial
  C. albicans PKH1 fragment
TIRRNVTDFKFGKELGEGSYSTVILATDKITGKQYAVKVLDKRHIIKEKKVKYVNIEKHA

SEQ ID NO: 18 - protein, 334 aa, Artificial
  PDK1-PKH1 chimera
MSYYHHHHHHDYDIPTTENLYFQGAMDGTAAEPRPGAGSLQHAQPPPQPRRKRPEDFKFGKILGEGSFSTVVLARELATSREYAIKILEKRHIIKENKVPYVNREKDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 19 - DNA, 2952 bp, Homo sapiens
  CDS (1)..(2952)
atggcgtccaaccccgaacggggggagattctgctcacggaactgcagggggattcccgaagtcttccgttttctgagaatgtgagtgctgttcaaaaattagacttttcagatacaatggtgcagcagaaattggatgatatcaaggatcgaattaagagagaaataaggaaagaactgaaaatcaaagaaggagctgaaaatctgaggaaagtcacaacagataaaaaaagtttggcttatgtagacaacattttgaaaaaatcaaataaaaaattagaagaactacatcacaagctgcaggaattaaatgcacatattgttgtatcagatccagaagatattacagattgcccaaggactccagatactccaaataatgaccctcgttgttctactagcaacaatagattgaaggccttacaaaaacaattggatatagaacttaaagtaaaacaaggtgcagagaatatgatacagatgtattcaaatggatcttcaaaggatcggaaactccatggtacagctcagcaactgctccaggacagcaagacaaaaatagaagtcatacgaatgcagattcttcaggcagtccagactaatgaattggcttttgataatgcaaaacctgtgataagtcctcttgaacttcggatggaagaattaaggcatcattttaggatagagtttgcagtagcagaaggtgcaaagaatgtaatgaaattacttggctcaggaaaagtaacagacagaaaagcactttcagaagctcaagcaagatttaatgaatcaagtcagaagttggaccttttaaagtattcattagagcaaagattaaacgaagtccccaagaatcatcccaaaagcaggattattattgaagaactttcacttgttgctgcatcaccaacactaagtccacgtcaaagtatgatatctacgcaaaatcaatatagtacactatccaaaccagcagcactaacaggtactttggaagttcgtcttatgggctgccaagatatcctagagaatgtccctggacggtcaaaagcaacatcagttgcactgcctggttggagtccaagtgaaaccagatcatctttcatgagcagaacgagtaaaagtaaaagcggaagtagtcgaaatcttctaaaaaccgatgacttgtccaatgatgtctgtgctgttttgaagctcgataatactgtggttggccaaactagctggaaacccatttccaatcagtcatgggaccagaagtttacactggaactggacaggtcacgtgaactggaaatttcagtttattggcgtgattggcggtctctgtgtgctgtaaaatttctgaggttagaagattttttagacaaccaacggcatggcatgtgtctctatttggaaccacagggtactttatttgcagaggttaccttttttaatccagttattgaaagaagaccaaaacttcaaagacaaaagaaaattttttcaaagcaacaaggcaaaacatttctcagagctcctcaaatgaatattaatattgccacttggggaaggctagtaagaagagctattcctacagtaaatcattctggcaccttcagccctcaagctcctgtgcctactacagtgccagtggttgatgtacgcatccctcaactagcacctccagctagtgattctacagtaaccaaattggactttgatcttgagcctgaacctcctccagccccaccacgagcttcttctcttggagaaatagatgaatcttctgaattaagagttttggatataccaggacaggattcagagactgtttttgatattcagaatgacagaaatagtatacttccaaaatctcaatctgaatacaagcctgatactcctcagtcaggcctagaatatagtggtattcaagaacttgaggacagaagatctcagcaaaggtttcagtttaatctacaagatttcaggtgttgtgctgtcttgggaagaggacattttggaaaggtgcttttagctgaatataaaaacacaaatgagatgtttgctataaaagccttaaagaaaggagatattgtggctcgagatgaagtagacagcctgatgtgtgaaaaaagaatttttgaaactgtgaatagtgtaaggcatccctttttggtgaacctttttgcatgtttccaaaccaaagagcatgtttgctttgtaatggaatatgctgccggtggggacctaatgatgcacattcatactgatgtcttttctgaaccaagagctgtattttatgctgcttgtgtagttcttgggttgcagtatttacatgaacacaaaattgtttatagagatttgaaattggataacttattgctagatacagagggctttgtgaaaattgctgattttggtctttgcaaagaaggaatgggatatggagatagaacaagcacattttgtggcactcctgaatttcttgccccagaagtattaacagaaacttcttatacaagggctgtagattggtggggccttggcgtgcttatatatgaaatgcttgttggtgagtctccctttcctggtgatgatgaagaggaagtttttgacagtattgtaaatgatgaagtaaggtatccaaggttcttatctacagaagccatttctataatgagaaggctgttaagaagaaatcctgaacggcgccttggggctagcgagaaagatgcagaggatgtaaaaaagcacccatttttccggctaattgattggagcgctctgatggacaaaaaagtaaagccaccatttatacctaccataagaggacgagaagatgttagtaattttgatgatgaatttacctcagaagcacctattctgactccacctcgagaaccaaggatactttcggaagaggagcaggaaatgttcagagattttgactacattgctgattggtgt

SEQ ID NO: 20 - protein, 984 aa, Homo sapiens
MASNPERGEILLTELQGDSRSLPFSENVSAVQKLDFSDTMVQQKLDDIKDRIKREIRKELKIKEGAENLRKVTTDKKSLAYVDNILKKSNKKLEELHHKLQELNAHIVVSDPEDITDCPRTPDTPNNDPRCSTSNNRLKALQKQLDIELKVKQGAENMIQMYSNGSSKDRKLHGTAQQLLQDSKTKIEVIRMQILQAVQTNELAFDNAKPVISPLELRMEELRHHFRIEFAVAEGAKNVMKLLGSGKVTDRKALSEAQARFNESSQKLDLLKYSLEQRLNEVPKNHPKSRIIIEELSLVAASPTLSPRQSMISTQNQYSTLSKPAALTGTLEVRLMGCQDILENVPGRSKATSVALPGWSPSETRSSFMSRTSKSKSGSSRNLLKTDDLSNDVCAVLKLDNTVVGQTSWKPISNQSWDQKFTLELDRSRELEISVYWRDWRSLCAVKFLRLEDFLDNQRHGMCLYLEPQGTLFAEVTFFNPVIERRPKLQRQKKIFSKQQGKTFLRAPQMNINIATWGRLVRRAIPTVNHSGTFSPQAPVPTTVPVVDVRIPQLAPPASDSTVTKLDFDLEPEPPPAPPRASSLGEIDESSELRVLDIPGQDSETVFDIQNDRNSILPKSQSEYKPDTPQSGLEYSGIQELEDRRSQQRFQFNLQDFRCCAVLGRGHFGKVLLAEYKNTNEMFAIKALKKGDIVARDEVDSLMCEKRIFETVNSVRHPFLVNLFACFQTKEHVCFVMEYAAGGDLMMHIHTDVFSEPRAVFYAACVVLGLQYLHEHKIVYRDLKLDNLLLDTEGFVKIADFGLCKEGMGYGDRTSTFCGTPEFLAPEVLTETSYTRAVDWWGLGVLIYEMLVGESPFPGDDEEEVFDSIVNDEVRYPRFLSTEAISIMRRLLRRNPERRLGASEKDAEDVKKHPFFRLIDWSALMDKKVKPPFIPTIRGREDVSNFDDEFTSEAPILTPPREPRILSEEEQEMFRDFDYIADWC

SEQ ID NO: 21 - protein, 84 aa, Artificial
  PDK1 fragment
PRKKRPEDFKFGKILGEGSFSTVVLARELATSREYAIKILEKRHIIKENKVPYVTRERDVMSRLDHPFFVKLYFTFQDDEKLYF

SEQ ID NO: 22 - protein, 87 aa, Artificial
  PRK2 fragment
RFQFNLQDFRCCAVLGRGHFGKVLLAEYKNTNEMFAIKALKKGDIVARDEVDSLMCEKRIFETVNSVRHPFLVNLFACFQTKEHVCF

SEQ ID NO: 23 - protein, 334 aa, Artificial
  PDK1-PRK2 chimera
MSYYHHHHHHDYDIPTTENLYFQGAMDGTAAEPRPGAGSLQHAQPPPQPRQKRPEDFKFGKILGEGSFSTVVLARELATSREYAIKILEKRHIVKENKVPYLMREKDVMSRLDHPFFVKLYFCFQDDEKVYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 24 - DNA, 1293 bp, Homo sapiens
  CDS (1)..(1293)
atgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctc

SEQ ID NO: 25 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 26 - protein, 77 aa, Artificial; identical to SEQ ID NO: 11
  PDK1 fragment

SEQ ID NO: 27 - protein, 78 aa, Artificial
  SGK1 fragment
NPHAKPSDFHFLKVIGKGSFGKVLLARHKAEEVFYAVKVLQKKAILKKKEEKHIMSERNVLLKNVKHPFLVGLHFSFQ

SEQ ID NO: 28 - protein, 334 aa, Artificial
  PDK1-SGK1 chimera
MSYYHHHHHHDYDIPTTENLYFQGAMDGTAAEPRPGAGSLQHAQPPPQPRHKRPEDFKFGKILGEGSFSTVVLARELATSREYAIKILEKKHILKENKEKYIMRERDVMSRLDHPFFVKLYFSFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 29 - DNA, 1575 bp, Homo sapiens
  CDS (1)..(1575)
atgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatcta

SEQ ID NO: 30 - protein, 525 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 31 - protein, 86 aa, Artificial
  PDK1 fragment
PRKKRPEDFKFGKILGEGSFSTVVLARELATSREYAIKILEKRHIIKENKVPYVTRERDVMSRLDHPFFVKLYFTFQDDEKLYFGL

SEQ ID NO: 32 - protein, 90 aa, Artificial
  S6K1 fragment
PEKIRPECFELLRVLGKGGYGKVFQVRKVTGANTGKIFAMKVLKKAMIVRNAKDTAHTKAERNILEEVKHPFIVDLIYAFQTGGKLYLIL

SEQ ID NO: 33 - protein, 334 aa, Artificial
  PDK1-S6K1 chimera
MSYYHHHHHHDYDIPTTENLYFQGAMDGTAAEPRPGAGSLQHAQPPPQPRKKRPEDFKFGKILGEGSFSTVVLARELATSREYAIKILEKRHIVKENKTPYTKRERDVMSRLDHPFFVKLYFAFQDDEKLYLGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 34 - DNA, 1440 bp, Homo sapiens
  CDS (1)..(1440)
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcc

SEQ ID NO: 35 - protein, 480 aa, Homo sapiens [sequence omitted]

SEQ ID NO: 36 - protein, 84 aa, Artificial; identical to SEQ ID NO: 21
  PDK1 fragment

SEQ ID NO: 37 - protein, 84 aa, Artificial
  AKT1 fragment
KHRVTMNEFEYLKLLGKGTFGKVILVKEKATGRYYAMKILKKEVIVAKDEVAHTLTENRVLQNSRHPFLTALKYSFQTHDRLCF

SEQ ID NO: 38 - protein, 334 aa, Artificial
  PDK1-AKT1 chimera
MSYYHHHHHHDYDIPTTENLYFQGAMDGTAAEPRPGAGSLQHAQPPPQPRRKRPEDFKFGKILGEGSFSTVVLARELATSREYAIKILEKEHIVKENKVPYTLRENDVMSRLDHPFFVKLYFSFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 39 - DNA, 1443 bp, Homo sapiens
  CDS (1)..(1443)
atgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcggaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgag

SEQ ID NO: 40 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 41 - protein, 84 aa, Artificial; identical to SEQ ID NO: 21
  PDK1 fragment

SEQ ID NO: 42 - protein, 84 aa, Artificial
  AKT2 fragment
RAKVTMNDFDYLKLLGKGTFGKVILVREKATGRYYAMKILRKEVIIAKDEVAHTVTESRVLQNTRHPFLTALKYAFQTHDRLCF

SEQ ID NO: 43 - protein, 334 aa, Artificial
  PDK1-AKT2 chimera
MSYYHHHHHHDYDIPTTENLYFQGAMDGTAAEPRPGAGSLQHAQPPPQPRKKRPEDFKFGKILGEGSFSTVVLARELATSREYAIKILEKEHIIKENKVPYTVRESDVMSRLDHPFFVKLYFAFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 44 - DNA, 2220 bp, Homo sapiens
  CDS (1)..(2220)
atgccgctggcgcagctggcggacccgtggcagaagatggctgtggagagcccgtccgacagcgctgagaatggacagcaaattatggatgaacctatgggagaggaggagattaacccacaaactgaagaagtcagtatcaaagaaattgcaatcacacatcatgtaaaggaaggacatgaaaaggcagatccttcccagtttgaacttttaaaagtattagggcagggatcatttggaaaggttttcttagttaaaaaaatctcaggctctgatgctaggcagctttatgccatgaaggtattgaagaaggccacactgaaagttcgagaccgagttcggacaaaaatggaacgtgatatcttggtagaggttaatcatccttttattgtcaagttgcattatgcttttcaaactgaagggaagttgtatcttattttggattttctcaggggaggagatttgtttacacgcttatccaaagaggtgatgttcacagaagaagatgtcaaattctacttggctgaacttgcacttgctttagaccatctacatagcctgggaataatttatagagacttaaaaccagaaaatatacttcttgatgaagaaggtcacatcaagttaacagatttcggcctaagtaaagagtctattgaccatgaaaagaaggcatattctttttgtggaactgtggagtatatggctccagaagtagttaatcgtcgaggtcatactcagagtgctgactggtggtcttttggtgtgttaatgtttgaaatgcttactggtacactccctttccaaggaaaagatcgaaaagaaacaatgactatgattcttaaagccaaacttggaatgccacagtttttgagtcctgaagcgcagagtcttttacgaatgcttttcaagcgaaatcctgcaaacagattaggtgcaggaccagatggagttgaagaaattaaaagacattcatttttctcaacgatagactggaataaactgtatagaagagaaattcatccgccatttaaacctgcaacgggcaggcctgaagatacattctattttgatcctgagtttactgcaaaaactcccaaagattcacctggcattccacctagtgctaatgcacatcagctttttcgggggtttagttttgttgctattacctcagatgatgaaagccaagctatgcagacagttggtgtacattcaattgttcagcagttacacaggaacagtattcagtttactgatggatatgaagtaaaagaagatattggagttggctcctactctgtttgcaagagatgtatacataaagctacaaacatggagtttgcagtgaagattattgataaaagcaagagagacccaacagaagaaattgaaattcttcttcgttatggacagcatccaaacattatcactctaaaggatgtatatgatgatggaaagtatgtgtatgtagtaacagaacttatgaaaggaggtgaattgctggataaaattcttagacaaaaatttttctctgaacgagaggccagtgctgtcctgttcactataactaaaaccgttgaatatcttcacgcacaaggggtggttcatagagacttgaaacctagcaacattctttatgtggatgaatctggtaatccggaatctattcgaatttgtgattttggctttgcaaaacagctgagagcggaaaatggtcttctcatgactccttgttacactgcaaattttgttgcaccagaggttttaaaaagacaaggctatgatgctgcttgtgatatatggagtcttggtgtcctactctatacaatgcttaccggttacactccatttgcaaatggtcctgatgatacaccagaggaaatattggcacgaataggtagcggaaaattctcactcagtggtggttactggaattctgtttcagacacagcaaaggacctggtgtcaaagatgcttcatgtagaccctcatcagagactgactgctgctcttgtgctcagacatccttggatcgtccactgggaccaactgccacaataccaactaaacagacaggatgcaccacatctagtaaagggtgccatggcagctacatattctgctttgaaccgtaatcagtcaccagttttggaaccagtaggccgctctactcttgctcagcggagaggtattaaaaaaatcacctcaacagccctg

SEQ ID NO: 45 - protein, 740 aa, Homo sapiens
MPLAQLADPWQKMAVESPSDSAENGQQIMDEPMGEEEINPQTEEVSIKEIAITHHVKEGHEKADPSQFELLKVLGQGSFGKVFLVKKISGSDARQLYAMKVLKKATLKVRDRVRTKMERDILVEVNHPFIVKLHYAFQTEGKLYLILDFLRGGDLFTRLSKEVMFTEEDVKFYLAELALALDHLHSLGIIYRDLKPENILLDEEGHIKLTDFGLSKESIDHEKKAYSFCGTVEYMAPEVVNRRGHTQSADWWSFGVLMFEMLTGTLPFQGKDRKETMTMILKAKLGMPQFLSPEAQSLLRMLFKRNPANRLGAGPDGVEEIKRHSFFSTIDWNKLYRREIHPPFKPATGRPEDTFYFDPEFTAKTPKDSPGIPPSANAHQLFRGFSFVAITSDDESQAMQTVGVHSIVQQLHRNSIQFTDGYEVKEDIGVGSYSVCKRCIHKATNMEFAVKIIDKSKRDPTEEIEILLRYGQHPNIITLKDVYDDGKYVYVVTELMKGGELLDKILRQKFFSEREASAVLFTITKTVEYLHAQGVVHRDLKPSNILYVDESGNPESIRICDFGFAKQLRAENGLLMTPCYTANFVAPEVLKRQGYDAACDIWSLGVLLYTMLTGYTPFANGPDDTPEEILARIGSGKFSLSGGYWNSVSDTAKDLVSKMLHVDPHQRLTAALVLRHPWIVHWDQLPQYQLNRQDAPHLVKGAMAATYSALNRNQSPVLEPVGRSTLAQRRGIKKITSTAL

SEQ ID NO: 46 - protein, 86 aa, Artificial; identical to SEQ ID NO: 31
  PDK1 fragment

SEQ ID NO: 47 - protein, 88 aa, Artificial
  RSK2 fragment
HEKADPSQFELLKVLGQGSFGKVFLVKKISGSDARQLYAMKVLKKATLKVRDRVRTKMERDILVEVNHPFIVKLHYAFQTEGKLYLIL

SEQ ID NO: 48 - protein, 334 aa, Artificial
  PDK1-RSK2 chimera
MSYYHHHHHHDYDIPTTENLYFQGAMDGTAAEPRPGAGSLQHAQPPPQPRKKRPEDFKFGKILGEGSFSTVVLARELATSREYAIKILEKRHTLKENKRPYTKRERDVMSRLDHPFFVKLYFAFQDDEKLYLGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 49 - DNA, 2406 bp, Homo sapiens
  CDS (1)..(2406)
atggaggaggagggtggcagcagcggcggcgccgcggggaccagcgcggacggcggcgacggaggagagcagctcctcactgtcaagcacgagctgcggactgctaatttgacaggacatgctgagaaggtgggaatagaaaattttgagctcctgaaggtcctaggaactggagcttatggaaaagtatttctagttcgtaaaataagtggccatgatactggaaagctgtatgccatgaaagttttgaaaaaggcaacaatcgttcaaaaggccaaaaccacagagcatacaaggacagaacgacaagtcctggaacacattaggcagtcgccatttttggtaacattacattatgctttccagacagaaaccaaacttcatctcattttagattatataaatggtggtgaactttttactcatctttctcaaagagagcgtttcacagagcatgaggtgcagatttatgttggagagattgtgcttgccctcgaacatctccacaagttggggattatatatcgtgatattaagcttgagaatattctacttgattctaatggccatgtggtgctgacagattttggtctgagtaaggagtttgtggctgatgaaactgaaagagcatattccttttgtggaactattgaatacatggcaccagatattgtcagagggggagattcaggacatgacaaggcagttgactggtggagtttgggtgttctaatgtatgaattactaactggagcatctcctttcactgttgatggagaaaaaaattcccaagctgagatatctaggagaatattaaaaagtgagcctccatatccccaagaaatgagtgctttagcgaaagacctaattcagcgtcttttgatgaaagatcccaagaagagattgggatgtggtccacgtgatgcagatgaaatcaaagaacatctcttctttcagaaaataaattgggatgatttagccgccaaaaaagtgcctgcaccatttaagccagtcattcgagatgaattagatgtgagtaactttgcagaagagttcacagaaatggatcccacttattctcccgcagccctgccccagagttctgagaagctgtttcagggctattcctttgttgctccttccatcctattcaagcgtaatgcagctgtcatagaccctcttcagtttcacatgggagttgaacgtcctggagtgacaaatgttgccaggagtgcaatgatgaaggactctccattctatcaacactatgacctagatttgaaggacaaacccctgggagaaggtagtttttcaatttgtcgaaagtgtgtgcataaaaaaagtaaccaagcttttgcagtcaaaataatcagcaaaaggatggaagccaatactcaaaaggaaataacagctctgaaactctgtgaaggacaccccaatattgtgaagttgcatgaagtttttcatgatcagcttcacacgtttctagtgatggaacttctgaatggaggagaactgtttgagcgcattaagaaaaagaagcacttcagtgagacggaagccagctacatcatgaggaagcttgtttcagctgtaagccacatgcatgatgttggagtggtgcacagggatctgaaacctgagaatttattgttcaccgatgaaaatgacaatttggaaattaaaataattgattttggatttgcacggctaaagccaccggataatcagcccctgaagactccatgcttcacccttcattatgccgccccagagctcttgaatcagaacggctacgatgagtcctgtgacctgtggagcttgggcgtcattttgtacacaatgttgtcaggacaggttcccttccaatctcatgaccgaagtttgacgtgtaccagcgcggtggaaatcatgaagaaaattaaaaagggagatttctcctttgaaggagaagcctggaagaatgtatcccaagaggctaaagatttgatccaaggacttctcacagtagatccaaacaaaaggcttaaaatgtctggcttgaggtacaatgaatggctacaagatggaagtcagctgtcctccaatcctctgatgactccggatattctaggatcttccggagctgccgtgcatacctgtgtgaaagcaaccttccacgcctttaacaaatacaagagagaggggttttgccttcagaatgttgataaggcccctttggctaagagaagaaaaatgaaaaagactagcaccagtaccgagacgcgcagcagttccagtgagagttcccattcttcttcctctcattctcacggtaaaactacacccaccaagacactgcagcccagcaatcctgccgacagcaataacccggagaccctcttccagttctcggactcagtagct

SEQ ID NO: 50 - protein, 802 aa, Homo sapiens
MEEEGGSSGGAAGTSADGGDGGEQLLTVKHELRTANLTGHAEKVGIENFELLKVLGTGAYGKVFLVRKISGHDTGKLYAMKVLKKATIVQKAKTTEHTRTERQVLEHIRQSPFLVTLHYAFQTETKLHLILDYINGGELFTHLSQRERFTEHEVQIYVGEIVLALEHLHKLGIIYRDIKLENILLDSNGHVVLTDFGLSKEFVADETERAYSFCGTIEYMAPDIVRGGDSGHDKAVDWWSLGVLMYELLTGASPFTVDGEKNSQAEISRRILKSEPPYPQEMSALAKDLIQRLLMKDPKKRLGCGPRDADEIKEHLFFQKINWDDLAAKKVPAPFKPVIRDELDVSNFAEEFTEMDPTYSPAALPQSSEKLFQGYSFVAPSILFKRNAAVIDPLQFHMGVERPGVTNVARSAMMKDSPFYQHYDLDLKDKPLGEGSFSICRKCVHKKSNQAFAVKIISKRMEANTQKEITALKLCEGHPNIVKLHEVFHDQLHTFLVMELLNGGELFERIKKKKHFSETEASYIMRKLVSAVSHMHDVGVVHRDLKPENLLFTDENDNLEIKIIDFGFARLKPPDNQPLKTPCFTLHYAAPELLNQNGYDESCDLWSLGVILYTMLSGQVPFQSHDRSLTCTSAVEIMKKIKKGDFSFEGEAWKNVSQEAKDLIQGLLTVDPNKRLKMSGLRYNEWLQDGSQLSSNPLMTPDILGSSGAAVHTCVKATFHAFNKYKREGFCLQNVDKAPLAKRRKMKKTSTSTETRSSSSESSHSSSSHSHGKTTPTKTLQPSNPADSNNPETLFQFSDSVA

SEQ ID NO: 51 - protein, 86 aa, Artificial; identical to SEQ ID NO: 31
  PDK1 fragment

SEQ ID NO: 52 - protein, 91 aa, Artificial
  MSK1 fragment
AEKVGIENFELLKVLGTGAYGKVFLVRKISGHDTGKLYAMKVLKKATIVQKAKTTEHTRTERQVLEHIRQSPFLVTLHYAFQTETKLHLIL

SEQ ID NO: 53 - protein, 334 aa, Artificial
  PDK1-MSK1 chimera
MSYYHHHHHHDYDIPTTENLYFQGAMDGTAAEPRPGAGSLQHAQPPPQPRKKRPEDFKFGKILGEGSFSTVVLARELATSREYAIKILEKRHIVKENKTEYTRRERDVMSRLDHPFFVKLYFAFQDDEKLYLGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEGLIFAKIIKLEYDFPEKFFPKARDLVEKLLVLDATKRLGCEEMEGYGPLKAHPFFESVTWENLHQQTPPKLT

SEQ ID NO: 54 - protein, 10 aa, Homo sapiens
AGNEYLIFQK

SEQ ID NO: 55 - protein, 8 aa, Homo sapiens
LDHPFFVK

SEQ ID NO: 56 - protein, 17 aa, Artificial
  PKCiota substrate
KSIYRRGSRRWRKLYRA

SEQ ID NO: 57 - protein, 16 aa, Artificial
  PDK1 substrate
KTFCGTPEYLAPEVRR

SEQ ID NO: 58 - protein, 18 aa, Artificial
  fragment of PDK1-PKCiota chimera
KSIYRRGARRWRKLYRAN

SEQ ID NO: 59 - protein, 17 aa, Artificial
  ROCK-HM
VGNQLPFIGFTYFRENL

SEQ ID NO: 60 - protein, 20 aa, Artificial
  activation loop of PKA
RTWALCGTPEYLAPEIILKK